(19) United States
(12) Patent Application Publication
(10) Pub. No.: US 2021/0341462 A1
(43) Pub. Date: Nov. 4, 2021

(54) ARTIFICIAL HUMAN PULMONARY AIRWAY AND METHODS OF PREPARATION

(71) Applicants: Dongeun HUH, Villanova, PA (US); Andrei GEORGESCU, Philadelphia, PA (US); THE TRUSTEES OF THE UNIVERSITY OF PENNSYLVANIA, Philadelphia, PA (US)

(72) Inventors: Dongeun Huh, Villanova, PA (US); Andrei Georgescu, Philadelphia, PA (US)

(73) Assignee: THE TRUSTEES OF THE UNIVERSITY OF PENNSYLVANIA, Philadelphia, PA (US)

(21) Appl. No.: 17/282,667

(22) PCT Filed: Oct. 7, 2019
(86) PCT No.: PCT/US2019/054986

§ 371 Date: Apr. 2, 2021

Related U.S. Application Data
(60) Provisional application No. 62/741,773, filed on Oct. 5, 2018.

(51) Int. Cl. G01N 33/50; C12N 5/071; C12N 5/00; C12M 3/06; C12M 1/00; C12M 1/12

(52) U.S. Cl. CPC G01N 33/5005; C12N 5/0697; C12N 5/0688; C12N 2513/00; C12M 23/16; C12M 23/34; C12M 25/14; C12N 5/0062

(57) ABSTRACT

The presently disclosed subject matter provides a microfluidic device that can simulate the cross section of the large and small human airways, including the air-exposed epithelial layer, the adjacent surrounding stromal layer, and the blood-facing endothelial layer of near-by vessels in the circulatory system. The microfluidic device can reconstitute the air-liquid interface in the lung and molecular transport characteristics of bronchi and bronchioles in the human pulmonary airways, and provide a more realistic alternative to current in vitro models of airway structures. Additionally, the model can reconstitute the native response of airway tissues to infection by bacterial and viral agents, and also the extravasation of immune cells from the bloodstream and into the stromal and epithelial compartments of the lung in response to an infection. The presently disclosed subject matter also provides microfluidic devices that include multiple chambers assembled by layered stacking or bonding of a basal chamber, a first membrane, an interstitial chamber, a second membrane and an apical chamber.

### CROSS-REFERENCE TO RELATED APPLICATIONS

This application claims priority to U.S. Provisional Application Ser. No. 62/741,773, filed Oct. 5, 2018, which is incorporated by reference herein in its entirety.

### STATEMENT OF GOVERNMENT INTEREST

This invention was made with government support under Award No. CASIS 1UG3TR002198-01 awarded by the National Institute of Health. The government has certain rights in the invention.

### BACKGROUND

The lungs are the principal components of the human respiratory system and are responsible for gas exchange: the absorption of oxygen from the air and into the bloodstream, and the release of carbon dioxide from the bloodstream and into the air. To reach the distal alveoli at which this gas exchange occurs, inhaled air flows through the pulmonary airways, which branch from the trachea (about 20-about 25 mm in diameter) into two primary bronchi (about 10-about 15 mm in diameter) to supply the left and right lungs, and then conducted further into secondary bronchi (about 5-about 10 mm in diameter) and tertiary bronchi (about 1-about 5 mm in diameter) that supply the lobes of each lung, and even further through multiple branchings of bronchioles (about 0.5-about 1 mm in diameter) and terminal bronchioles (about 0.3-about 0.5 mm diameter).

An apparent function of these bronchi and bronchioles is to form a branched tree architecture that efficiently conducts air to each alveoli, but they also perform additional duties: they modify their diameter via bronchoconstriction and bronchodilation processes to modify lung airflow in response to triggers including physical activity, temperature, and the presence of allergens, and they clear the lungs of foreign objects and debris through a process called mucociliary clearance, in which the beating of cilia on the air-facing epithelial cells transports mucus (and debris, bacteria, viruses, and foreign objects contained therein) towards the mouth.

The bronchi and bronchioles can be supplied with nutrients and oxygen by capillaries of the bronchial circulatory system, which is separate from the pulmonary circulation that supplies the alveoli with blood for gas exchange.

Certain models of healthy and diseased large and small pulmonary airways have been created to aid in fundamental investigations into the nature of pulmonary biology as well as disease or injury, and to develop physiologically relevant models for therapeutic applications including drug screening. However, such models have been limited in their capacity to reconstitute human lung physiology; certain in vivo animal models, for example, can be limited due to interspecies differences in lung physiology and behavior, and traditional 2D monolayer culture of human airway cells in both submerged and Transwell formats do not recapitulate the complex three-dimensional structure and dynamic mechanical and biochemical environment of the human lung. Additionally, certain microphysiological models of lung focus on mimicking the alveolar lining and barrier function thereof, and do not account for the effects of the stromal and interstitial tissue between the airways and bloodstream, nor for the behaviors and biochemical contributions of the cells therein, in both healthy or diseased states.

Therefore, there is a need for a low-cost, human cell-alternative to model of the human lung. Additionally, there is a need for a model that can reconstitute the physiological mechanical and biochemical environment of the human pulmonary airways to the extent required to model dynamic behaviors including responses to various kinds of external and internal stimuli including the effects from smoking, mechanical ventilation, total/partial liquid ventilation, indwelling biomedical devices, clinical diagnostic procedures, radiation, tissue engineering scaffolds, or inhalation of particulates, environmental toxins, occupational and environmental chemicals, aerosolized drugs, cells, pathogens; the development and effects of abnormal lung tissue remodeling, development, regeneration, and injuries; as well as the dynamics of lung infections and inflammation and the resulting recruitment of immune cells in response to such a challenge.

### SUMMARY

The presently disclosed subject matter provides microfluidic devices adapted to function as a low-cost, human cell-alternative to model the human lung, or animal-cell alternatives to experimentation on living animals. In certain example embodiments, the device includes a basal chamber, an apical chamber and a central interstitial chamber. The basal chamber includes a first microfluidic channel disposed thereon, the central interstitial chamber includes a second microfluidic channel disposed thereon, and the apical chamber includes a third microfluidic channel disposed thereon. The device also includes a first membrane disposed between the basal chamber and the interstitial chamber, a second membrane disposed between the interstitial chamber and the apical chamber, a three-dimensional, extracellular matrix hydrogel disposed in the interstitial chamber between the first and second membranes, and a base supporting the first, second and third microfluidic channels. The microfluidic device can also include support pillars to prevent membrane deflection. In certain embodiments, the microfluidic device can contain multiple interstitial chambers stacked vertically to create layered structures reminiscent of stromal tissues in the lung. In certain embodiments, more than one interstitial chamber can be used and bonded to the intervening membranes. In certain embodiments, more than one apical chamber can be used and bonded to the second membrane. In certain microfluidic embodiments, more than one basal chamber can be used and bonded to the first membrane.

In certain embodiments, human primary lung fibroblast cells are encapsulated within a hydrogel scaffold in the interstitial chamber. In certain embodiments, immune cells (e.g., macrophages, monocytes, dendritic cells, etc.) are encapsulated within a hydrogel. In certain embodiments, vascular cells can be encapsulated within a hydrogel. In certain embodiments, adipose cells can be encapsulated within a hydrogel. In certain embodiments, muscle cells can be encapsulated within a hydrogel. In certain embodiments, cartilage cells can be encapsulated within a hydrogel. In certain embodiments, cancer cells can be encapsulated within a hydrogel. In certain embodiments, stem cells can be encapsulated within a hydrogel.

In certain embodiments, normal cells are cultured in the chambers. In certain embodiments, diseased cells are cultured in the chambers. In certain embodiments, a combination of normal and diseased cells are cultured in the chambers.

In certain embodiments, cells disposed into the subject matter are primary human cells. In certain embodiments, cells disposed into the subject matter are primary animal cells. In certain embodiments, cells disposed into the subject matter are immortalized cell lines. In certain embodiments, cells disposed into the subject matter are stem cells. In certain embodiments, cells disposed into the subject matter are embryonic stem cells. In certain embodiments, cells disposed into the subject matter are induced pluripotent stem cells (iPSCs). In certain embodiments, cells disposed into the subject matter are a combination of primary human cells, primary animal cells, cell lines, stem cells, and induced pluripotent stem cells.

In certain embodiments, the basal chamber(s) is in fluid and physical communication with the interstitial chamber(s) through the first membrane. In certain embodiments, the interstitial chamber(s) is in fluid and physical communication with the apical chamber(s) through the second membrane. In certain embodiments, the basal chamber(s), the interstitial chamber(s) and the apical chamber(s) can be in fluid and physical communication through the first membrane and the second membrane. In certain embodiments, the interstitial chamber(s) is continuous between the first and second membranes such that fluid and physical communication is permitted between the basal chamber(s) and the apical chamber(s) via the interstitial chamber(s). In certain embodiments, the first membrane has a first monolayer of epithelial cells disposed thereon, and the second membrane has a second monolayer of epithelial cells disposed thereon. In certain embodiments, the first membrane has a monolayer of epithelial cells disposed thereon, and the second membrane has a monolayer of endothelial cells disposed thereon.

The first and the second membranes can be associated with endothelial and epithelial cells, respectively. However, in certain embodiments, for example, to enhance imaging in an inverted microscope setup, the cells can be inversely oriented, with the epithelial cells facing downwards at the bottom of the microfluidic device, and the endothelial cells at the top of the device facing upwards. In this case, the topmost chamber would be called the “basal chamber” and the bottom chamber would be called the “apical chamber.” Additionally, in certain embodiments, in microfluidic devices with more than three chambers (as exemplified in in FIGS. 9B-9D), additional cell types can be present in different spatial configurations to model different tissue systems, or multiple layers of the human airway with increased granularity. For example, in certain embodiments, a 5-chamber system that is separated at each chamber-to-chamber interface by a membrane can contain the first and fifth (topmost and bottommost, respectively) chambers as endothelial cell chambers (basal chambers), the second and fourth chambers as interstitial chambers containing different densities of hydrogels or fibroblast cells, and the third, center chamber as the apical chamber with epithelial cells seeded onto both facing membranes. In certain embodiments, the absorption of toxic particulates flowing from the air-filled center chamber and into two different interstitial conditions (e.g., fibrotic vs. non-fibrotic) from one single airway lumen, for example, can then be modelled. In certain embodiments, different cell types, cells from different donors, cells cultured under different biochemical or mechanical conditions, cells administered different drug or environmental treatments, or a combination thereof can be cultured together in devices with 3 chambers, devices with more chambers (as a nonlimiting example, the 5-chamber orientation described in this paragraph), or devices with fewer chambers (as a nonlimiting example, in instances where the experiment is focused on the dynamics of just one compartment or chamber).

In certain embodiments, the first microchannel includes a basal microfluidic inlet port and a basal chamber outlet port disposed thereon, or an injection and an outlet port disposed thereon; the second microchannel includes an interstitial chamber injection port disposed thereon; and the third microchannel includes an apical microfluidic inlet port and an apical chamber outlet port disposed thereon. The first, second and third microchannels introduce a fluid to at least one or more of the basal, interstitial, and apical chambers.

In certain embodiments containing greater than 3 chambers, for example the 5-chamber device described in Paragraph 13, each chamber can contain a distinct microfluidic channel, and each microfluidic channel can contain an inlet, an outlet, or an inlet and an outlet. In certain embodiments, these microchannels that corresponding to chambers can be used to fill the chambers with fluid (including without limitation cell culture media, water, solvents, or a mixture of biological components) or with gases (including without limitation atmospheric air, a specific pre-mixed gas mixture, or a pure gas).

In certain embodiments, bidirectional fluid communication and species transport is permitted from the basal chamber, through the first membrane, through the interstitial chamber, and through the second membrane into the apical chamber.

In certain embodiments, more than one interstitial chambers are present and layered directly on top of each other. In certain embodiments, more than one interstitial chambers are present and layered such that a perfusable chamber is disposed between hydrogel-filled interstitial chambers. In certain embodiments, the perfusable chambers are filled with cell culture growth medium. In certain embodiments, cell culture growth medium-filled perfusable chambers between hydrogel-containing interstitial chambers are used to provide nutrients to the interstitial hydrogel-scaffolded tissue to overcome diffusional constraints in bulk hydrogels. In certain embodiments, the diffusional constraints limit hydrogel thickness to about 200 micrometers. In certain embodiments, diffusional constraints limit hydrogel thickness to about 1 millimeter. In certain embodiments, reduced diffusional constraints limit hydrogel thickness to about 5-10 millimeters. In certain embodiments, the pore size of semiporous membranes limits nutrient diffusion from cell culture media through hydrogels. In certain embodiments, nutrient diffusion from cell culture growth media-containing chambers into interstitial chambers forms a concentration gradient. In certain embodiments, a concentration gradient of nutrients is used to physiologically model the diffusion of nutrients into bulk tissue from blood vessels.

In certain embodiments, perfusable chambers are disposed between interstitial chambers containing hydrogels. In certain embodiments, vasculogenic combinations of cells including but not limited to endothelial cells, fibroblasts, mesenchymal stem cells, vascular pericyte cells, astrocyte cells, and tissue-specific cells are disposed into an interstitial chamber and allowed to form vessels over a period ranging from about 4 hours to about 4 months, depending on the biological simulation required by the subject matter. In certain embodiments, apposing perfusable chambers are used to deliver cell culture growth media to vasculogenic cells in the interstitial chambers. In certain embodiments, once vasculogenic cells in the interstitial chambers have formed perfusable tubule structures or vessel-like structures, the perfusable chambers are filled with cell-free hydrogels, or with cell-laden hydrogels, or with a combination of cell-free and cell-laden hydrogels, to form a continuous interstitial chamber possessing a total thickness amounting to the sum of the thicknesses of the individual hydrogel-filled chambers. In certain embodiments, the perfusable character of the tubule structures or vascular vessel-like structures created by cells in interstitial chambers laden with vasculogenic combinations of cells is used to circulate cell culture growth media through the interstitial chambers. In certain embodiments, circulation of cell culture growth media through tubule or vessel-like structures in interstitial chambers can be used to provide nutrients to initially perfusable, fluid-filled chambers that were subsequently filled with cell-free or cell-laden hydrogels.

In certain embodiments, multiple compositions of interstitial tissue can be layered between the basal and apical chambers. In certain embodiments, the compositions can use different concentrations of fibroblast cells to model pathophysiological conditions including fibrosis. In certain embodiments, the compositions can use different hydrogel stiffnesses to model a stiffness gradient from the epithelial tissue in the lung to the endothelial layer of a blood vessel. In certain embodiments, the compositions can vary in hydrogel crosslink density, in hydrogel chemical composition, in hydrogel chemical modifications, in hydrogel chemical modifications that enhance or disrupt cell behaviors including but not limited to attachment, motility, morphology, pathology, or biological pathway activation (in a non-limiting example, binding motifs in the hydrogel that initiate a cellular transduction cascade, or hormones or cytokines bound to the hydrogel to initiate physiological cellular-level or tissue-level responses).

The presently disclosed subject matter also provides methods of fabricating a microfluidic device having one or more basal chambers, one or more interstitial chambers, and one or more apical chambers. In an example embodiment, the method includes inserting a first membrane between the basal chamber and the interstitial chamber; inserting a second membrane between the interstitial chamber and the apical chamber; placing cells encapsulated in a pre-gel solution into the interstitial chamber; allowing a first monolayer of cells to grow on the first membrane; and allowing a second monolayer of cells to grow on the second membrane. The basal chamber can have a first microfluidic channel disposed thereon, the interstitial chamber can have a second microfluidic channel disposed thereon, and the apical chamber can have a third microfluidic channel disposed thereon.

In certain embodiments, the microfluidic device can be constructed without the intervening membranes between chambers by bonding the apical and basal chambers directly to the interstitial chambers.

The presently disclosed subject matter also provides methods of investigating the response of the pulmonary airway to an infection. An example method includes placing bacteria, viral capsids, or bacterial/viral products/derivatives/conditioned media in one or more apical chambers; allowing the pathogens to adhere to an epithelial layer in the apical chamber(s); placing white blood cells into one or more basal chambers; inverting the device to permit white blood cell adhesion to the endothelial cell monolayer on the surface of the first membrane facing the one or more basal chambers; monitoring white blood cell migration through the interstitial tissue to access the epithelial layer; and monitoring interactions of white blood cells with bacteria or with virus-infected cells. In certain embodiments, the method further includes allowing the virus to infect the cells. In certain embodiments, the method further includes inverting the device to permit white blood cell adhesion to the first membrane in the basal chamber(s).

The presently disclosed subject matter also provides methods of investigating the response of the pulmonary airway to environmental particulates, occupational hazards, hyperbaric or hypobaric atmospheres, hyperoxic or hypoxic atmospheres, or toxins, for acute exposures, chronic exposures or a combination of acute and chronic exposures. An exemplary method for purposes of illustration and not limitation includes flowing aerosolized particles, chemical vapors, or cigarette smoke in an apical chamber; allowing the species to interact with an epithelial layer in the apical chamber; placing white blood cells into a basal chamber; inverting the device to permit white blood cell adhesion to the endothelial cell layer in the basal chamber; monitoring white blood cell migration through the interstitial tissue to access the epithelial layer; monitoring epithelial injuries; monitoring epithelial barrier function; monitoring epithelial production of free radicals; monitoring endothelial activation; monitoring production of chemokines and cytokines by the epithelial cells and immune cells embedded in the interstitial chamber; monitoring remodeling of tissue layer in the interstitial chamber; and monitoring endothelial barrier permeability. In certain embodiments, the method further includes inverting the device to permit white blood cell adhesion to the first membrane in the basal chamber.

The presently disclosed subject matter also provides methods of investigating the response of the pulmonary airway to intratracheally administered drugs. An example method includes flowing aerosolized or dry powder drugs in an apical chamber; allowing the compounds to interact with a layer of diseased epithelial cells derived from chronic inflammatory lung diseases in the apical chamber; placing white blood cells into a basal chamber; inverting the device to permit white blood cell adhesion to the endothelial cell layer in the basal chamber; monitoring white blood cell migration through the interstitial tissue to access the epithelial layer; monitoring epithelial viability; monitoring epithelial production of free radicals; monitoring endothelial activation; monitoring production of chemokines and cytokines by the epithelial cells and immune cells embedded in the interstitial chamber; and monitoring remodeling of tissue layer in the interstitial chamber. In certain embodiments, the method further includes inverting the device to permit white blood cell adhesion to the first membrane in the basal chamber. In certain embodiments, gravitational settling is used to enhance white blood cell adhesion to a desired membrane, by orienting the device in a manner that positions the white blood cells above the desired membrane and allowing them to settle.

The presently disclosed subject matter also provides methods of investigating the response of the pulmonary airway to intravascularly administered drugs. An example method includes flowing chemotherapeutic drugs in an basal chamber; allowing the compounds to interact with a layer of endothelial cells in the basal chamber; placing white blood cells into a basal chamber; inverting the device to permit white blood cell adhesion to the endothelial cell layer in the basal chamber; monitoring white blood cell migration through the interstitial tissue to access the epithelial layer; monitoring endothelial barrier function; monitoring endothelial activation; monitoring endothelial viability; monitoring epithelial viability; monitoring epithelial production of free radicals; monitoring production of chemokines and cytokines by the epithelial cells and immune cells embedded in the interstitial chamber; and monitoring remodeling of tissue layer in the interstitial chamber.

The presently disclosed subject matter also provides methods of sampling or collecting the fluidic effluent of the basal or apical chambers, or from multiple chambers concurrently. In certain embodiments, this effluent can be collected once. In certain embodiments, this effluent can be collected at multiple sequential points. In certain embodiments, the state of the biological or fluidic elements of the subject matter can dictate the time at which the effluent is collected; in a non-limiting example, a fluid sample can be collected when transmitted light microscopy of cells indicates a physiological or morphological change, or a change that can suggest decreased viability. In a non-limiting example, a fluid sample can be collected when fluorescent microscopy of cells using a live/dead staining process or other fluorescent staining assessment including a free-radical staining or apoptotic pathway-activation staining indicates a change in the physiological or morphological state of the cells in the device. In certain embodiments, chemical analysis or biosensing can be performed on discrete sample(s) of collected effluent. In certain embodiments, continuous chemical analysis or biosensing can be performed on effluent flowing from a continuously perfused chamber. In certain embodiments, continuous chemical analysis or biosensing can be performed on fluidic effluent flowing from multiple continuously perfused chambers.

In certain embodiments, fluid containing secretory products from other biological sources, including but not limited to in vitro cell culture models, microphysiological systems, cellular monolayers, or in vivo sources including human or animal samples can be flowed into an apical or basal chamber, or into multiple chambers. In certain embodiments, the flow of fluid containing secretory products from other biological sources can be used to model the subject matter as a tissue or organ system as an element within a more expansive biological system, including but not limited to a “body on a chip” system in which the subject matter fulfills the role of a pulmonary model. In a non-limiting example, white blood cells from a human or animal can be introduced into an embodiment of the subject matter that has been infected with bacteria, to characterize the ability of those white blood cells to combat the infection.

In certain embodiments, the presently disclosed subject matter also provides methods of investigating the response of the pulmonary airway including the nasal cavity to disease states. In non-limiting examples, inflammation, age-related conditions, idiopathic conditions including idiopathic pulmonary fibrosis, genetic conditions, off-target drug effects, fibrosis, target drug effects, acute conditions, chronic conditions, or a combination thereof can be investigated in the subject matter. In certain embodiments, investigation into disease states can be performed by exposing the tissues cultured in the presently disclosed subject matter to the conditions responsible for disease onset. In certain embodiments, investigation into disease states can be performed by culturing patient-specific tissues or patient-specific cells obtained from patients affected by a disease or a combination of diseases targeted for investigation.

In certain embodiments, the presently disclosed subject matter also provides methods of investigating the response of the pulmonary airway including the nasal cavity to rare conditions for which there is no known animal model or experimental analog. In certain embodiments, the presently disclosed subject matter permits users to perform such experiments on human tissues without exposing human or animal subjects to physical or moral hazard. In certain embodiments, the presently disclosed subject matter can be used to investigate the effect of radioactive materials on biological tissues. In certain embodiments, the presently disclosed subject matter can be used to investigate the effects of inorganic materials and/or organic materials on human tissues. In certain embodiments, the presently disclosed subject matter can be used to investigate the effects of extraterrestrial materials including dust or foreign atmospheric samples on human tissues. In certain embodiments, the presently disclosed subject matter can be used to investigate the effects of human or animal exposure to Moon dust or Martian regolith.

In certain embodiments, the presently disclosed subject matter also provides methods of investigating the response of the pulmonary airway to electromagnetic or high-energy radiation. In certain embodiments, the effects on human or animal airway tissue from exposure to radiation from space travel, medical instrumentation, use of consumer products, or a combination thereof can be assessed.

In certain embodiments, the presently disclosed subject matter can be used to develop artificial pulmonary systems for extracorporeal substitutes or extracorporeal models of pulmonary function for living subjects. In certain embodiments, the artificial pulmonary systems can be used to replace the same or similar organ-specific functions. In certain embodiments, the artificial pulmonary systems can be used to monitor subject health preceding, during, or following an injury, disease, or other pathology or pathologic agent.

In certain embodiments, fluid containing secretory products from the presently disclosed subject matter can be collected from the apical chamber, the basal chamber, or from multiple chambers, and flowed or disposed onto or into other biological systems, including but not limited to in vitro cell culture models, microphysiological systems, cellular monolayers, or in vivo subjects including humans or animals. In certain embodiments, the flow of fluid containing secretory products from the presently disclosed subject matter into other biological subjects can be used to model the subject matter as a tissue or organ system as an element within a more expansive biological system, including but not limited to a “body on a chip” system in which the subject matter fulfills the role of a pulmonary model.

In certain embodiments, the presently disclosed subject matter can be deployed in a high throughput configuration for applications including but not limited to pharmaceutical screening. In certain embodiments, multiple copies or replicates of the presently disclosed subject matter can be distributed or stacked in a one-dimensional line or row, a two-dimensional grid or array, or a three-dimensional volume. In certain embodiments, multiple copies of the presently disclosed subject matter can be exposed to different conditions. In certain embodiments, morphological changes as a result of different experimental conditions can be assessed and compared. In certain embodiments, the assessment and analysis of morphological changes resulting from different experimental conditions can be used to infer biological processes or changes thereto including but not limited to gene expression, metabolism, pathology, and local or systemic communication.

In certain embodiments, the assessment and analysis of transcriptional changes or changes to the transcriptome resulting from different experimental conditions can be used to infer biological processes or changes thereto including but not limited to gene expression, metabolism, pathology, and local or systemic communication. In certain embodiments, the assessment and analysis of translational changes or changes to the proteome resulting from different experimental conditions can be used to infer biological processes or changes thereto including but not limited to gene expression, metabolism, pathology, and local or systemic communication. In certain embodiments, the assessment and analysis of secreted products or changes to the secretome from different experimental conditions can be used to infer biological processes or changes thereto including but not limited to gene expression, metabolism, pathology, and local or systemic communication. In certain embodiments, the assessment and analysis of changes to immune system recruitment, white blood cell recruitment, or inflammatory responses resulting from different experimental conditions can be used to infer biological processes or changes thereto including but not limited to gene expression, metabolism, pathology, and local or systemic communication. In certain embodiments, assessment of biological responses in the cells and tissues formed in the subject matter can be used to assess the efficacy of pharmaceutical compounds.

In certain embodiments, the devices can be imaged by an assortment of scientific modalities, including without limitation optical imaging, microscopic imaging, imaging by electron microscopy, imaging by high-energy radiation including X-rays, imaging by medical modalities including computed tomography (CT) and magnetic resonance imaging (MRI), or by a combination thereof.

In certain embodiments, the contents or a subset of the contents of the devices can be physically extracted from the device or from one or more of the chambers in the device. In certain embodiments, extraction of materials or biological tissues from the device facilitates analysis. In certain embodiments, extraction of materials or biological tissues from the device facilitates analysis by providing better access to the extracted specimen during imaging or sample processing applications and procedures.

### BRIEF DESCRIPTION OF THE FIGURES

FIGS. 1A and 1B illustrate alternative methods for modeling human large and small pulmonary airways for physiological and pathological studies or experimental investigations.

FIG. 2 illustrates a cross-sectional view of an exemplary microfluidic device model of a human pulmonary airway.

FIGS. 3A-3F provide exemplary microfluidic devices in accordance with the disclosed subject matter.

FIGS. 4A-4E illustrate a microfluidic device wherein epithelial cells can be fluorescently stained for occluding DAPI and F-actin, fibroblasts can be fluorescently stained with calcein AM, and endothelial cells can be fluorescently stained for DAPI and F-actin. FIG. 4D illustrates an expanded view of the microfluidic device in FIG. 4A.

FIG. 4B illustrates an expanded view of the hydrogel layer in FIG. 4D. FIG. 4C illustrates an expanded view of the epithelium in FIG. 4D. FIG. 4E illustrates an expanded view of the endothelium in FIG. 4D.

FIGS. 5A-5C illustrate the differentiation of the epithelial cell monolayer grown in an example device. FIG. 5B and FIG. 5C illustrate an expanded view of the epithelium in FIG. 5A.

FIG. 6 provides an exemplary method of fabricating a microfluidic device in accordance with the disclosed subject matter.

FIG. 7 provides an exemplary method of testing bacterial infection of the small airway in accordance with the disclosed subject matter.

FIG. 8 provides an exemplary method of testing the effects of toxins or particulates on the airway model in accordance with the disclosed subject matter.

FIGS. 9A-9D illustrate cross-sectional views of exemplary configurations of the disclosed microfluidic device.

FIG. 10 illustrates a certain embodiment of the microfluidic device in which multiple replicates of the microfluidic device have been deployed in a 2D grid within the standard footprint of a laboratory plate.

FIG. 11 provides an exemplary method of sampling fluids from the microfluidic devices in order to perform chemical or biological analysis, in accordance with the disclosed subject matter.

FIG. 12 provides an exemplary method of adding fluids from biological sources to the microfluidic device, in order to simulate the integration of the tissues modeled in the device with a larger biological system, e.g., as an entity within a “body on a chip” system, or as a means to test the ability of patient-derived white blood cells to test the function of those cells, in accordance with the disclosed subject matter.

FIG. 13 provides an exemplary method of sampling fluids from the microfluidic devices in order to simulate those fluids as having originated in the human lung, e.g., for subsequent delivery downstream to tissues within, e.g., a “body on a chip system,” in accordance with the disclosed subject matter.

FIG. 14 provides an exemplary method of deploying the microfluidic device into high-throughput configurations, in accordance with the disclosed subject matter.

FIG. 15A provides a top view of an exemplary microfluidic device in which one replicate of the microfluidic device is deployed.

FIG. 15B provides a top view of an exemplary microfluidic device in which multiple replicates of the microfluidic device have been deployed along a 1D row.

FIG. 15C provides a top view of an exemplary microfluidic device in which multiple replicates of the microfluidic device have been deployed along a 2D grid.

FIG. 15D provides a perspective view of an exemplary microfluidic device in which multiple replicates of the microfluidic device have been stacked into a 3D array.

FIG. 16 provides an exemplary method of extracting cultured cells, tissues, or materials cultured within an embodiment of the artificial pulmonary model, in accordance to the disclosed subject matter.

FIG. 17 illustrates an exemplary scanning electron micrograph of differentiated human primary small airway epithelial cells, whereby the micrograph was obtained in accordance with the method presented in FIG. 16.

FIG. 18 illustrates an exemplary scanning electron micrograph obtained in accordance with the method provided in FIG. 16, of differentiated human primary small airway epithelial cells infected with Pseudomonas aeruginosa bacteria, in a certain nonlimiting embodiment of the disclosed subject matter used in an exemplary fashion for in vitro disease modeling of lung infection.

FIG. 19 illustrates an exemplary scanning electron micrograph obtained in accordance with the method provided in FIG. 16, from a nonlimiting embodiment of small airway tissue cultured in accordance to the disclosed subject matter, illustrating without limitation the differentiation of exemplary human epithelial cells to form polarized cell bodies that are ciliated on their apical, air-facing surface.

FIG. 20 illustrates an exemplary transmission electron micrograph obtained in accordance with the method provided in FIG. 16, from a nonlimiting embodiment of small airway tissue cultured in accordance to the disclosed subject matter, illustrating without limitation the contact between multiple adjacent human airway epithelial cells linked by tight junction formation in a terminally differentiated epithelium.

FIG. 21 provides an exemplary method of extracting cultured cells or tissues cultured within an embodiment of the artificial pulmonary model for therapeutic applications including personalized medicine in certain nonlimiting embodiments.

FIG. 22 provides an exemplary method of extracting tissue products or materials cultured within an embodiment of the artificial pulmonary model for therapeutic applications including personalized medicine in certain nonlimiting embodiments.

FIG. 23 provides an exemplary method of extracting tissue products or materials that are cultured within an embodiment of the artificial pulmonary model, in such a way that the subject matter serves as a bioreactor for biological material production.

FIG. 24 illustrates an exemplary micrograph of extracellular matrix material extracted from the subject matter, in accordance with the method provided in FIG. 23.

### DETAILED DESCRIPTION

The subject matter disclosed herein leverages various microengineering technologies to develop a microengineered cell culture platform capable of reconstituting the three-dimensional microarchitecture, dynamic microenvironment, and physiological or pathological function of the human large and small pulmonary airways. In certain embodiments, the microfluidic device disclosed herein can allow for compartmentalized co-culture of human epithelial cells, lung fibroblasts, and pulmonary microvascular endothelial cells in a manner that simulates the complex architecture spanning the air-facing epithelial layer of an airway, the adjacent stromal tissue in the lung, and the vascular endothelium of a nearby bronchial blood vessel or capillary. In non-limiting embodiments, the microfluidic device can further simulate, in response to a bacterial or viral infection of the large or small airways, the extravasation of white blood cells from the bloodstream through the endothelium and into the lung interstitium, and the subsequent migration of the extravasated white blood cells towards or through the airway epithelium and towards the bacterial or viral infection. In certain embodiments, physiological flow conditions can be simulated in the system to mimic capillary blood flow beneath the endothelium. In certain embodiments, the physiological air-liquid interface conditions can be simulated in the system to mimic the air-facing epithelium, extracellular matrix interstitium, and endothelial lining of a blood capillary in the human lung. In certain embodiments, the physiological airflow conditions can be simulated in the system to mimic the sinusoidal flow of air along the airways in the lungs with each breath.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 15%, up to 10%, up to 5%, or up to 1% of a given value. Alternatively, particularly with respect to biological systems or processes, the term can mean an order of magnitude, preferably within five-fold, and more preferably within two-fold, of a value.

The terms “comprise(s),” “include(s),” “having,” “has,” “can,” “contain(s),” and variants thereof, as used herein, are intended to be open-ended transitional phrases, terms, or words that do not preclude additional acts or structures. The singular forms “a,” “an” and “the” include plural references unless the context clearly dictates otherwise. The present disclosure also contemplates other embodiments “comprising,” “consisting of”, and “consisting essentially of,” the embodiments or elements presented herein, whether explicitly set forth or not.

A “subject” herein can be a human or a non-human animal, for example, but not by limitation, rodents such as mice, rats, hamsters, and guinea pigs; rabbits; dogs; cats; sheep; pigs; goats; cattle; horses; and non-human primates such as apes and monkeys, etc.

In the detailed description herein, references to “embodiment,” “an embodiment,” “one embodiment”, “in various embodiments,” etc., indicate that the embodiment(s) described can include a particular feature, structure, or characteristic, but every embodiment might not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. After reading the description, it will be apparent to one skilled in the relevant art(s) how to implement the disclosure in alternative embodiments.

The term “pre-gel” as used herein refers to a solution composed in some part by extracellular matrix, monomers of a pre-polymer, partially crosslinked polymers that have not yet formed a hydrogel or a solid phase, or a combination thereof. In certain embodiments, this “pre-gel” solution is sufficiently inviscid that it may be injected or disposed into a chamber or onto a surface, membrane, or chamber. In certain embodiments, this “pre-gel” solution reacts chemically by covalent crosslinking, ionic interactions, or other chemically stabilizing schema to form a hydrogel or polymer phase from the formerly liquid “pre-gel” solution. For purposes of illustration and not limitation, a freshly mixed solution of fibrinogen and thrombin is considered a “pre-gel” solution; this solution can be injected, handled, or manipulated as a liquid, and after some time, this mixture forms a crosslinked hydrogel that is no longer a bulk liquid, which may serve in certain non-limiting embodiments as a cell scaffold, extracellular matrix, a substrate to mimic an extracellular matrix, a substrate to form a mechanical seal or septum between one or a plurality of chambers, a temporary substrate to form a mechanical interface prior to re-dissolution into a liquid phase, or a combination thereof. In certain embodiments, the “pre-gel” solution contains cells. In certain embodiments, the “pre-gel” solution contains biological factors including without limitation enzymes, growth factors, antibodies, lipids, drugs, or a combination thereof. In certain embodiments, the “pre-gel” solution contains nonbiological materials including without limitation salts, solvents, small molecules, dissolved metals or metal oxides, nanoparticles or microparticles, or a combination thereof. In certain embodiments, the “pre-gel” solution contains a combination of biological factors and nonbiological factors. In certain embodiments, the “pre-gel” solution forms a hydrogel in response to a change in temperature. In certain embodiments, the “pre-gel” solution forms a hydrogel in response to a change in pH. In certain embodiments, the “pre-gel” solution forms a hydrogel in response to mixing with a chemical initiator, which in certain non-limiting embodiments is a free-radical based crosslinking initiator, and in certain non-limiting embodiments is a photo-catalyzed chemical crosslinking initiator. In certain embodiments, the “pre-gel” solution forms a hydrogel after reacting for some period of time; in certain embodiments, this period of time is 10 seconds; in certain embodiments, this period of time is 30 minutes; in certain embodiments, this period of time ranges between 10 seconds and 30 minutes; in certain embodiments, this period of time is below 10 seconds or greater than 30 minutes. In certain embodiments, this period of time can be procedurally tuned. In certain embodiments, the “pre-gel” solution forms a hydrogel in response to a combination of all or a subset of the following nonlimiting factors: changes in temperature, changes in pH, use of a chemical initiator, or by reacting for some period of time.

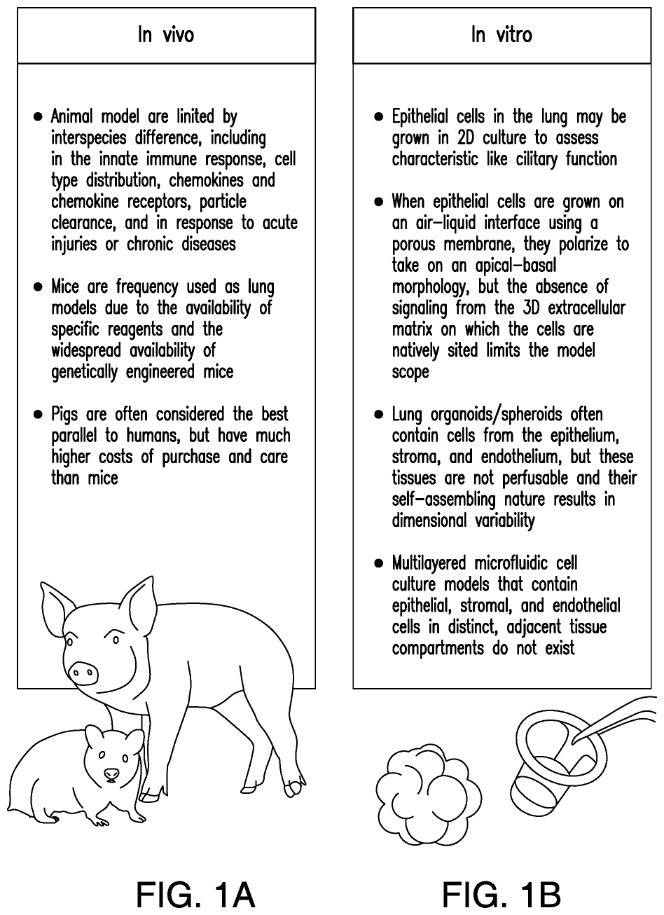

With reference to FIG. 1A for the purpose of illustration and not limitation, there is provided a schematic illustrating the limitations of using mice and pigs in lung model in vivo studies modeling human large and small pulmonary airways. With reference to FIG. 1B for the purpose of illustration and not limitation, there is provided a schematic illustrating that epithelial cells in the lung can be grown in 2D cultures to assess characteristics such as ciliary function used for in vitro studies modeling human large and small pulmonary airways.

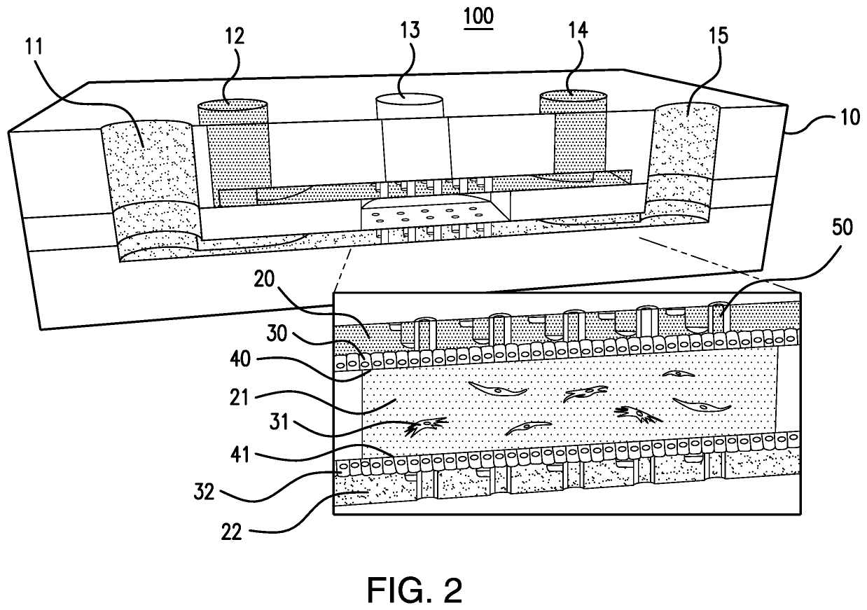

With reference to FIG. 2 for the purpose of illustration and not limitation, there is provided a schematic of an exemplary microfluidic device. In certain embodiments, the microfluidic device 100 can include a base 10, an apical chamber 20 (also referred to herein as an epithelial chamber, epithelium or as the chamber containing an epithelial layer), an interstitial chamber 21 (also referred to herein as interstitium, an interstitial layer, a stromal layer, or a hydrogel layer), a basal chamber 22 (also referred to herein as an endothelial chamber, endothelium or as the chamber containing an endothelial layer), a first membrane 41 with a first monolayer of endothelial cells 32 disposed thereon, a second membrane 40 with a second monolayer of epithelial cells 30 disposed thereon, an interstitial extracellular matrix hydrogel with encapsulated human primary lung fibroblast cells encapsulated therein 31, and support pillars 50 to prevent membrane deflection. The layer of epithelial cells resides on the surface of the second membrane that is facing the apical chamber. The layer of endothelial cells resides on the surface of the first membrane that faces the basal chamber.

In certain embodiments, the base 10 can have at least one or more microfluidic channels consisting of a first microfluidic channel, a second microfluidic channel and a third microfluidic channel. The first microfluidic channel includes a basal microfluidic inlet port 11 and a basal chamber outlet port 15 disposed thereon. These “microfluidic channels” 11 and 15 can be inlet/outlet ports as well (orientation of inlet and outlet can be reversed). The second microfluidic channel includes an interstitial chamber injection port 13. The third microfluidic channel includes an apical microfluidic inlet port 12 and an apical chamber outlet port. 14.

The microfluidic channels can have any suitable dimensions. For example, in certain embodiments, the cross-sectional size of the microfluidic channels can be about 10 mm (width)×about 10 mm (length)×about 1 mm (height). In certain embodiments, the microfluidic channels can be about 200 μm (width)×about 200 μm (length)×about 100 μm (height). In certain embodiments, the microfluidic channels can be about 1.5 cm in length. In certain embodiments, the cross-sectional size of the microfluidic channels can have different dimensions. Other channels at this scale (several microns to several millimeters) through which a fluid can be flowed can be microfluidic channels. The apical and basal chambers are also “microfluidic channels,” as are the conduits that connect these chambers to the fluid ports a short distance away.

In certain embodiments, looking top down, the apical, interstitial, and basal chambers can have a footprint of about 4 mm by about 4 mm. In certain embodiments, the apical, interstitial, and basal chambers can have a footprint of about 2 mm by about 2 mm. In certain embodiments, the apical, interstitial, and basal chambers can have a footprint of about 10 mm by about 10 mm or higher.

In certain embodiments, looking from the side at a cross section, the apical chamber can be about 1 mm tall. In certain embodiments, the apical chamber can be in the range of from about 100 μm to about 2000 μm tall. The basal chamber is the same size as the apical chamber.

In certain embodiments, looking from the side at a cross section, the interstitial chamber can be about 250 μm tall. In certain embodiments, the interstitial chamber can be smaller (e.g., about 50 μm tall) to simulate a very small gap between epithelium and endothelium. In certain embodiments, the interstitial chamber can be thicker (e.g., about 1000 μm) to simulate a larger distance between epithelium and endothelium, or to simulate the pathophysiology of e.g., lung fibrosis.

Connected to these chambers are microfluidic channels as follows: an inlet channel and an outlet channel for the apical layer, and inlet channel and an outlet channel for the basal layer, and an inlet channel (or inlet channel and outlet channel) for the interstitial layer. Media is flowed inward through the inlet channel and out through the outlet channel in the apical and basal chambers, and the hydrogel solution containing stromal cells is disposed in the interstitial chamber through the inlet microchannel. In certain embodiments, in order to fit several devices close together for throughput, the length of the inlet and outlet microfluidic channels can be reduced. In certain embodiments, the length of the inlet and outlet microfluidic channels can be lengthened.

In certain embodiments, the membrane 41 can be disposed between basal chamber 22 and interstitial chamber 21 such that the basal chamber 22 and the interstitial chamber 21 can be in fluid communication through the membrane 41. In certain embodiments, the membrane 40 can be disposed between interstitial chamber 21 and apical chamber 20 such that the interstitial chamber 21 and the apical chamber 20 can be in fluid communication through the membrane 40. In certain embodiments, the first membrane 41 can be disposed between basal chamber 22 and interstitial chamber 21 and the second membrane 40 can be disposed between interstitial chamber 21 and apical chamber 20, such that the basal chamber 22, the interstitial chamber 21, and the apical chamber 20 can be in fluid communication through the first membrane 41 and the second membrane 40. In certain embodiments, the membranes 40 and 41 can be a thin polyester membrane and can have 0.4 μm pores. In certain embodiments, the pores can be any suitable size. In certain embodiments, the membrane can include porous portions and nonporous portions. In certain embodiments, the membranes 40 and 41 can be a polycarbonate membrane, a polyester membrane, a polytetrafluoroethylene membrane, an elastomeric membrane, a plastic membrane, a paper membrane, an extracellular matrix membrane, or any other suitable membrane. The selection of the pore sizes, materials, and other features of the membrane can be varied based on the design of the microfluidic device, and the experimental goals, or other suitable motivations.

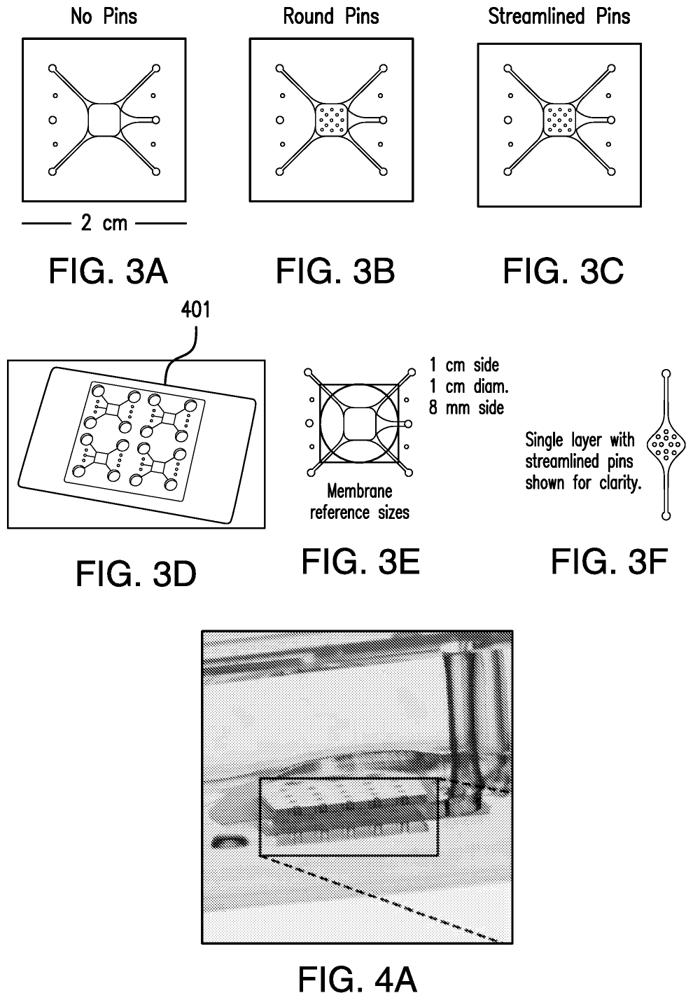

In certain embodiments, the base 10 can include multiple groups of chambers, each group acting as an independent microfluidic device, to facilitate high-throughput experiments. In certain embodiments, multiple groups of chambers can be connected to facilitate fluid placement, collection, outflow, or manipulation by, e.g., connecting apical outlet port 14 on a first microfluidic device to apical inlet port 12 on a second device such that a stream of air or fluid can be flowed through both chambers sequentially, from only one air or fluid source. In certain embodiments, the microfluidic device can have the schematic design shown in FIG. 3. In certain embodiments, four microfluidic airway devices can be disposed in the base 401.

With reference to FIG. 3A for the purpose of illustration and not limitation, there is provided an illustration of an exemplary microfluidic device with no pins with a dimension of about 2 cm per side.

With reference to FIG. 3B for the purpose of illustration and not limitation, there is provided an illustration of an exemplary microfluidic device with round pins.

With reference to FIG. 3C for the purpose of illustration and not limitation, there is provided an illustration of an exemplary microfluidic device with streamlined pins.

With reference to FIG. 3D for the purpose of illustration and not limitation, there is provided an illustration of an exemplary microfluidic device 401 with four repeating units.

With reference to FIG. 3E for the purpose of illustration and not limitation, there is provided an illustration of an exemplary microfluidic device with membrane reference dimensions of about a 1 cm side, about a 1 cm diameter and about an 8 mm side.

With reference to FIG. 3F for the purpose of illustration and not limitation, there is provided an illustration of a single layer exemplary microfluidic device with streamlined pins.

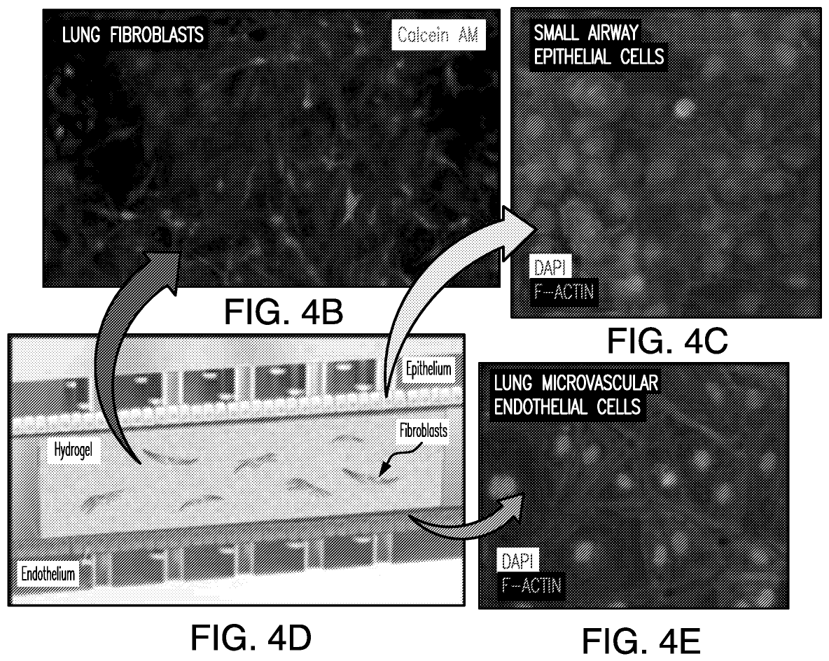

With reference to FIG. 4A for the purpose of illustration and not limitation, there is provided an illustration of the epithelium, hydrogel and endothelium layers in an exemplary microfluidic device. With reference to FIG. 4D for the purpose of illustration and not limitation, there is provided an expanded view of the microfluidic device of FIG. 4A.

With reference to FIG. 4B for the purpose of illustration and not limitation, there is provided an expanded view of the hydrogel layer of FIG. 4D. As shown in FIG. 4B, the lung fibroblasts can be fluorescently stained with calcein AM.

With reference to FIG. 4C for the purpose of illustration and not limitation, there is provided an expanded view of the epithelium in FIG. 4D. As shown in FIG. 4C, the small airway epithelial cells can be fluorescently stained for DAPI and F-actin.

With reference to FIG. 4E for the purpose of illustration and not limitation, there is provided an expanded view of the endothelium in FIG. 4D. As shown in FIG. 4E, the lung microvascular endothelial cells can be fluorescently stained for DAPI and F-actin.

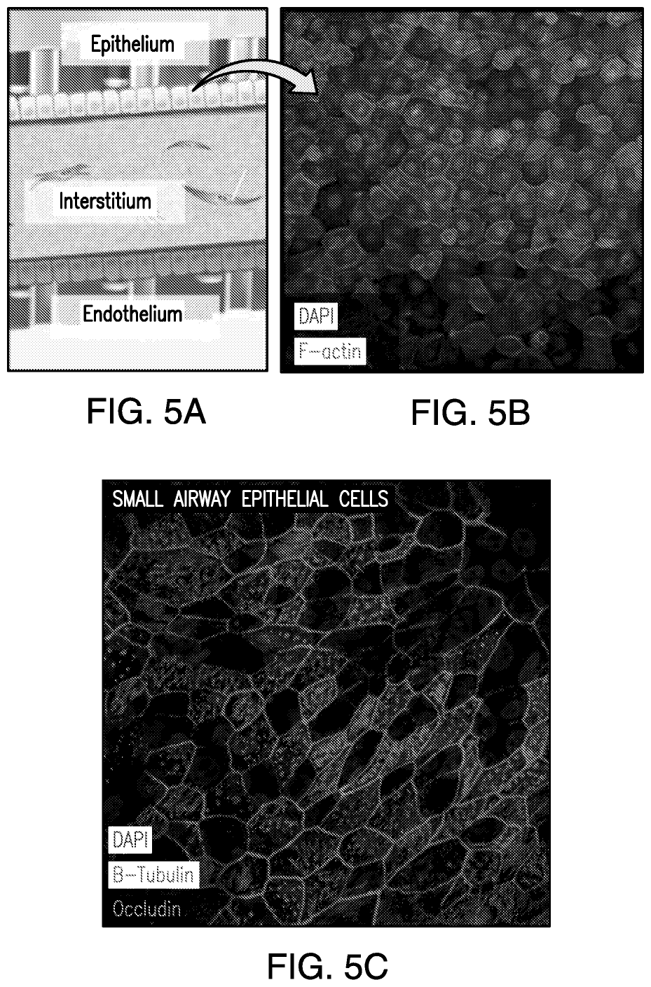

With reference to FIG. 5A for the purpose of illustration and not limitation, there is provided an illustration of the epithelium, interstitium and endothelium layers in an exemplary microfluidic device.

With reference to FIG. 5B for the purpose of illustration and not limitation, there is provided an expanded view of the epithelium in FIG. 5A. FIG. 5B shows the differentiation of the epithelial cell monolayer grown in an exemplary microfluidic device, which has been fluorescently stained for DAPI and F-actin.

With reference to FIG. 5C for the purpose of illustration and not limitation, there is provided an expanded view of the epithelium in FIG. 5A. FIG. 5C shows the differentiation of the epithelial cell monolayer grown in an exemplary microfluidic device, which has been fluorescently stained for occludin, DAPI and beta-tubulin.

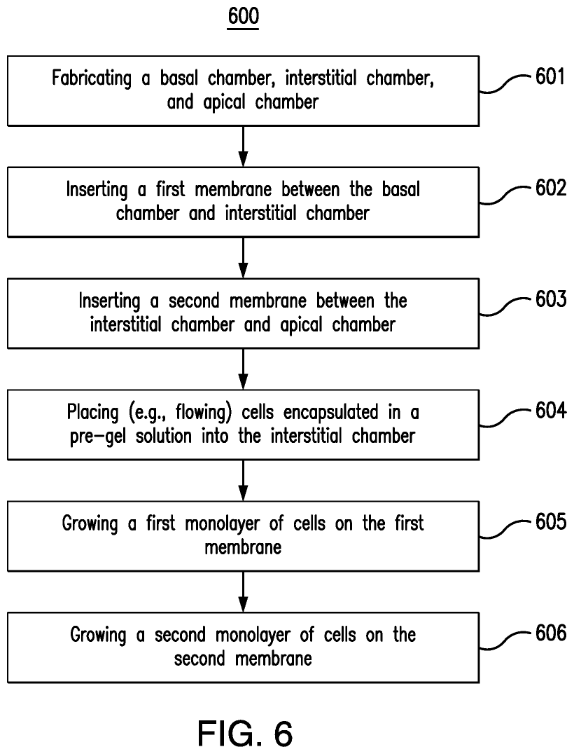

With reference to FIG. 6 for the purpose of illustration and not limitation, there is provided an exemplary method of fabricating a microfluidic device 600. As shown in FIG. 6, the method shows at 601 that a basal chamber, an interstitial chamber, and an apical chamber can be fabricated. At 602, a first membrane can be inserted between the basal chamber and the interstitial chamber. At 603, a second membrane can be inserted between the interstitial chamber and the apical chamber. At 604, cells encapsulated in a pre-gel solution can be placed into the interstitial chamber. At 605, a first monolayer of cells can be grown on the first membrane. At 606, a second monolayer of cells can be grown on the second membrane.

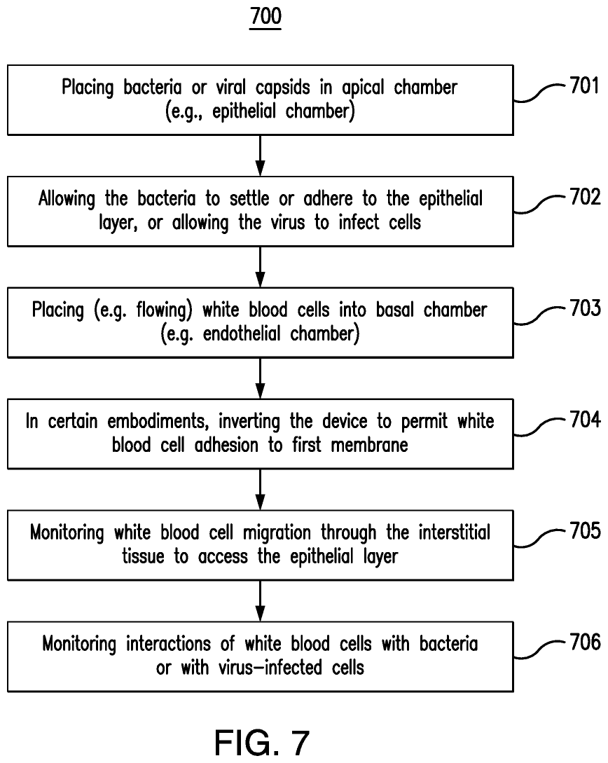

With reference to FIG. 7 for the purpose of illustration and not limitation, there is provided an exemplary method of testing bacterial infection of the small airway of the lung using an exemplary microfluidic device 700. As shown in FIG. 7, the method shows at 701 that bacteria or viral capsids can be placed into the apical chamber. At 702, the bacteria can be allowed to adhere to the epithelial layer. At 702, the virus can be allowed to infect the cells. At 703, the white blood cells can be placed into the basal chamber. At 704, in certain embodiments, the device can be inverted to permit white blood cell adhesion to the first membrane. At 705, the white blood cell migration through the interstitial tissue to the epithelial layer can be monitored. At 706, the interactions of the white blood cells with bacteria or with virus-infected cells can be monitored. The white blood cells are added to the basal chamber, which has endothelial cells lining the surface of the first membrane (the surface which faces the basal chamber). In certain embodiments, the microfluidic device described herein can be similarly used for testing bacterial and/or viral infection of the large airway of the lung and/or the nasal cavity.

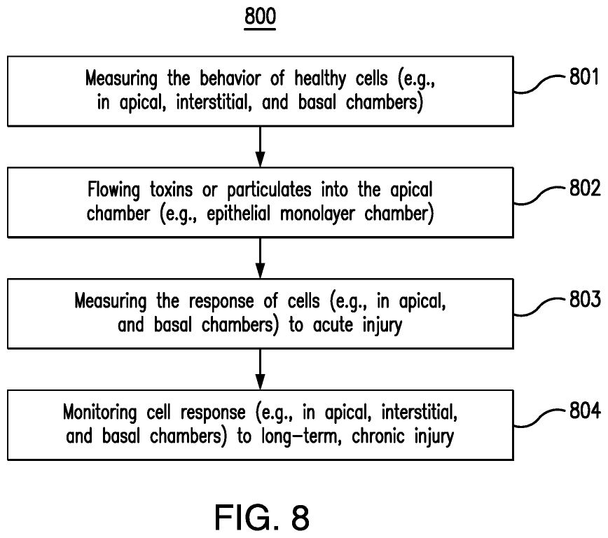

With reference to FIG. 8 for the purpose of illustration and not limitation, there is provided an exemplary method of testing the effects of toxins or particulates on the airway model using an exemplary microfluidic device 800. As shown in FIG. 8, the method shows in 801 that the behavior of healthy cells in the apical, interstitial and basal chambers can be measured. At 802, toxins or particulates can be placed into the apical chamber, for example, into the chamber containing epithelial cells on the second membrane. At 803, the response of the cells to acute injury, for example, in the apical and basal chambers, can be measured. At 804, the cell response to long-term, chronic injury, for example, the cells in the apical and basal chambers, can be monitored.

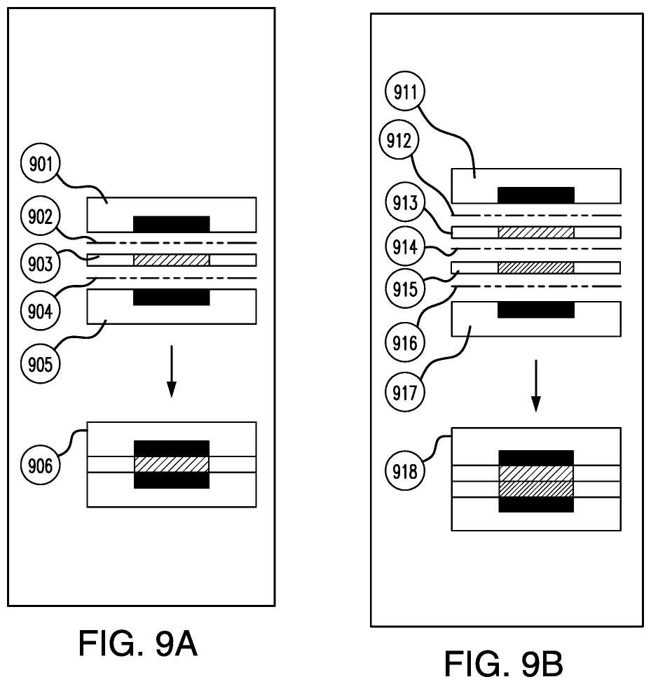

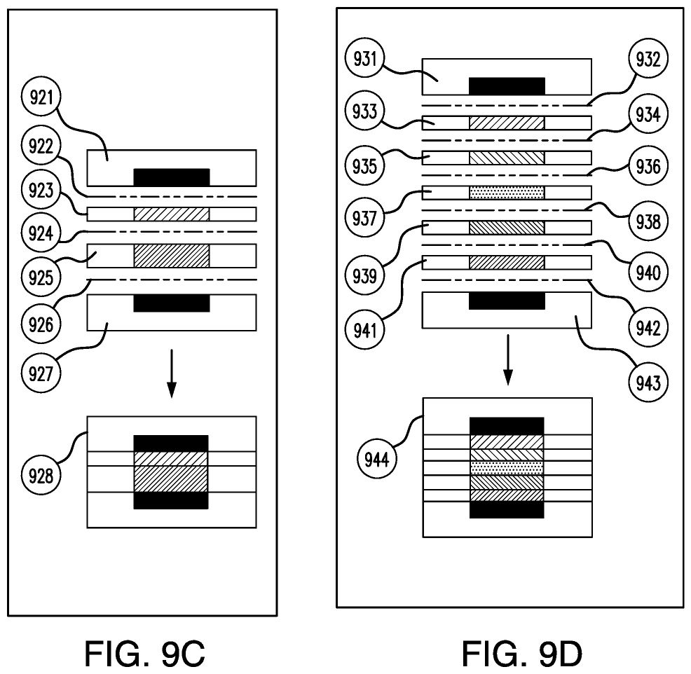

With reference to FIG. 9A for the purpose of illustration and not limitation, there is provided a cross-sectional view of an exemplary 3-chamber microfluidic device 906 assembled by layered stacking or bonding of a basal chamber 905, a first membrane 904, an interstitial chamber 903, a second membrane 902, and an apical chamber 901.

With reference to FIG. 9B for the purpose of illustration and not limitation, there is provided a cross-sectional view of an exemplary 4-chamber microfluidic device 918 assembled by layered stacking or bonding of a basal chamber 917, a first membrane 916, a first interstitial chamber 915, a second membrane 914, a second interstitial chamber 913, a third membrane 912, and an apical chamber 911.

With reference to FIG. 9C for the purpose of illustration and not limitation, there is provided a cross-sectional view of an exemplary 4-chamber microfluidic device 928 assembled by layered stacking or bonding of a basal chamber 927, a first membrane 926, a first interstitial chamber 925, a second membrane 924, a second interstitial chamber 923 of different thickness than the first interstitial chamber 925, a third membrane 922, and an apical chamber 921.

With reference to FIG. 9D for the purpose of illustration and not limitation, there is provided a cross-sectional view of an exemplary 7-chamber microfluidic device 944 assembled by layered stacking or bonding of a basal chamber 943, a first membrane 942, a first interstitial chamber 941, a second membrane 940, a second interstitial chamber 939, a third membrane 938, a third interstitial chamber 937, a fourth membrane 936, a fourth interstitial chamber 935, a fifth membrane 934, a fifth interstitial chamber 933, a sixth membrane 932, and an apical chamber 931.

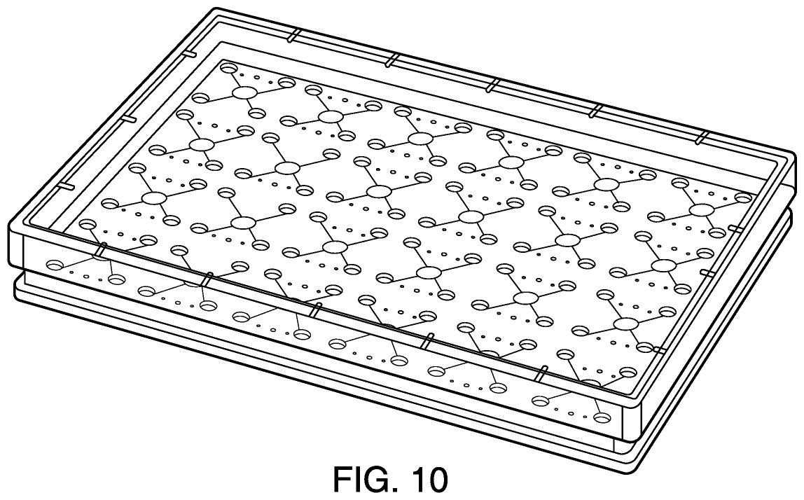

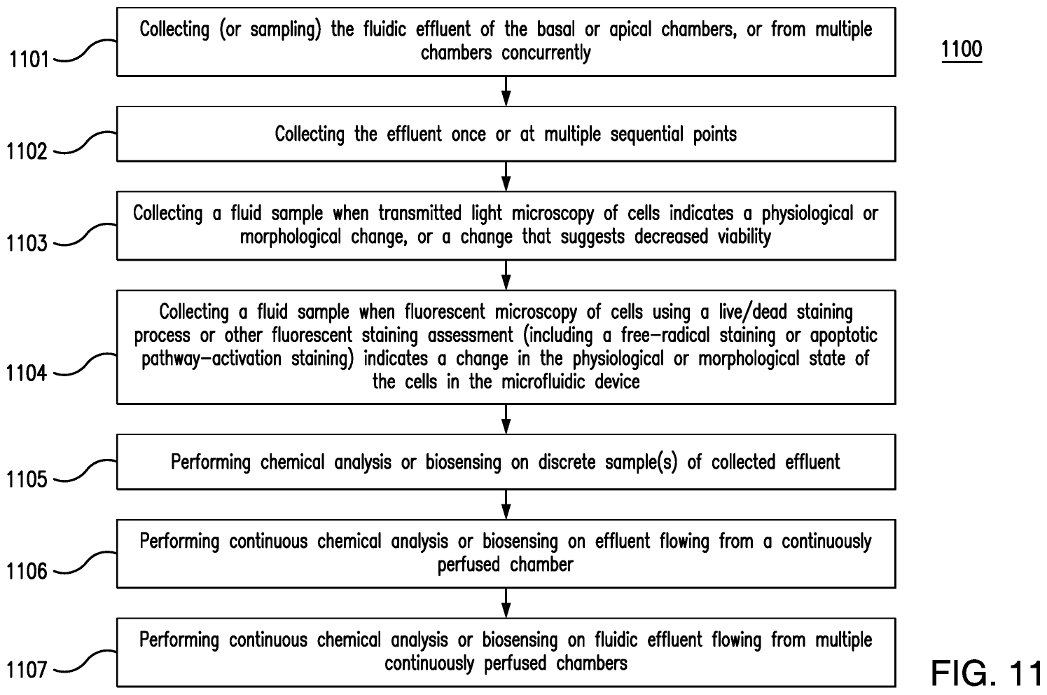

With reference to FIG. 10, for the purpose of illustration and not limitation, there is provided an exemplary method of deploying the microfluidic device in a high-throughput configuration contained within a typical plate for laboratory automation or handling. In such a high throughput plate, individual replicates of the microfluidic device can be distributed in a 1-dimensional row, in a 2-dimensional grid, placed in a 3-dimensional stack in order to facilitate experiments included but not limited to those that require multiple conditions to be examined. In certain embodiments, an array of replicates can contain 2 or more replicates. In certain embodiments, an array of replicates can contain about 10-about 1,000 replicates in a plate. In certain embodiments, more than about 1,000 replicates can be distributed within a plate. In certain embodiments, 3D stacking of additional replicates can provide additional room for replicate placement. In certain embodiments, the size of features in the subject matter can be adjusted to allow more or fewer replicates to fit within a given plate or array footprint.

With reference to FIG. 11 for the purpose of illustration and not limitation, there is provided an exemplary method of sampling fluids from the microfluidic device 1100. As shown in FIG. 11, the method shows at 1101 that the fluidic effluent of the basal or apical chambers, or from multiple chambers can be collected or sampled concurrently. At 1102, the effluent can be collected once or at multiple sequential points. At 1103, a fluid sample can be collected when transmitted light microscopy of cells indicates a physiological or morphological change, or a change that suggests decreased viability. At 1104, a fluid sample can be collected when fluorescent microscopy of cells using a live/dead staining process or other fluorescent staining assessment, including a free-radical staining or apoptotic pathway-activation staining, indicates a change in the physiological or morphological state of the cells in the microfluidic device. At 1105, chemical analysis or biosensing can be performed on discrete sample(s) of collected effluent. At 1106, chemical analysis or biosensing can be performed on effluent flowing from a continuously perfused chamber. At 1107, chemical analysis or biosensing can be performed on fluidic effluent flowing from multiple continuously perfused chambers.

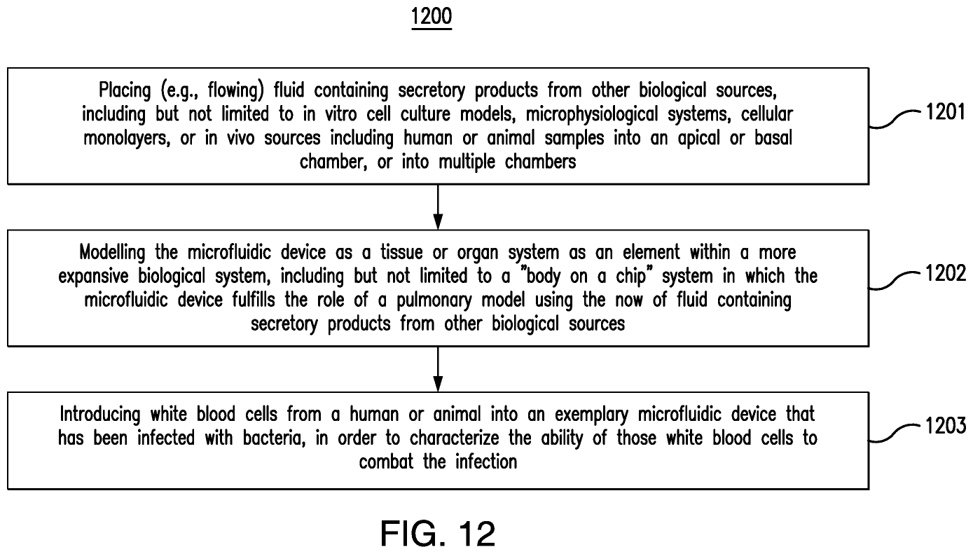

With reference to FIG. 12 for the purpose of illustration and not limitation, there is provided an exemplary method of adding fluids from biological sources to microfluidic device 1200. As shown in FIG. 12, the method shows at 1201 that fluid containing secretory products from other biological sources, including but not limited to in vitro cell culture models, microphysiological systems, cellular monolayers, or in vivo sources including human or animal samples can be placed (or flowed) into an apical or basal chamber, or into multiple chambers.

At 1202, the flow of fluid containing secretory products from other biological sources can be used to model the microfluidic device as a tissue or organ system as an element within a more expansive biological system, including but not limited to a “body on a chip” system in which the microfluidic device fulfills the role of a pulmonary model. At 1203, white blood cells from a human or animal can be introduced into an exemplary microfluidic device that has been infected with bacteria, to characterize the ability of those white blood cells to combat the infection.

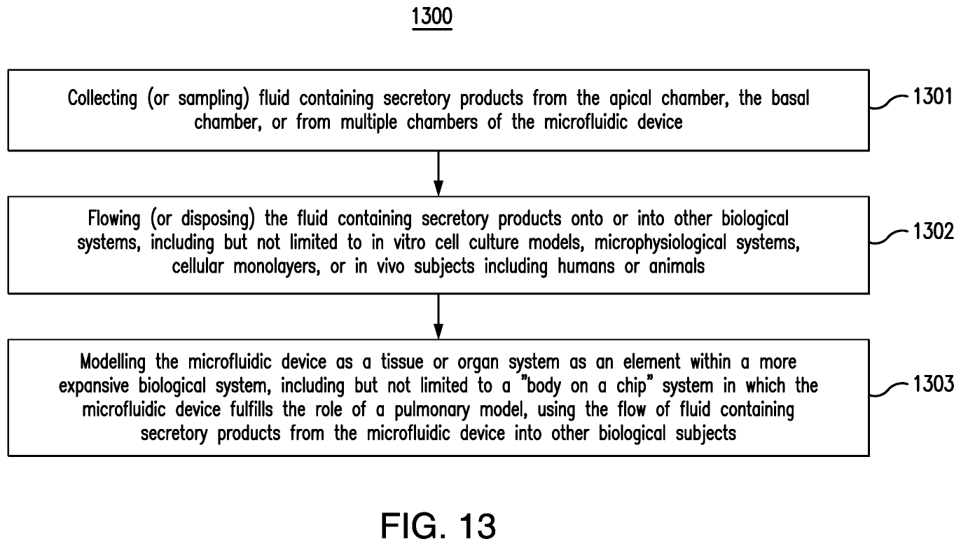

With reference to FIG. 13 for the purpose of illustration and not limitation, there is provided an exemplary method of sampling fluids from microfluidic device 1300. As shown in FIG. 13, the method shows at 1301 that fluid containing secretory products from the microfluidic device can be collected (or sampled) from the apical chamber, the basal chamber, or from multiple chambers. At 1302, the fluid containing secretory products can be flowed (or disposed) onto or into other biological systems, including but not limited to in vitro cell culture models, microphysiological systems, cellular monolayers, or in vivo subjects including humans or animals. At 1303, the flow of fluid containing secretory products from the microfluidic device into other biological subjects can be used to model the microfluidic device as a tissue or organ system as an element within a more expansive biological system, including but not limited to a “body on a chip” system in which the microfluidic device fulfills the role of a pulmonary model.

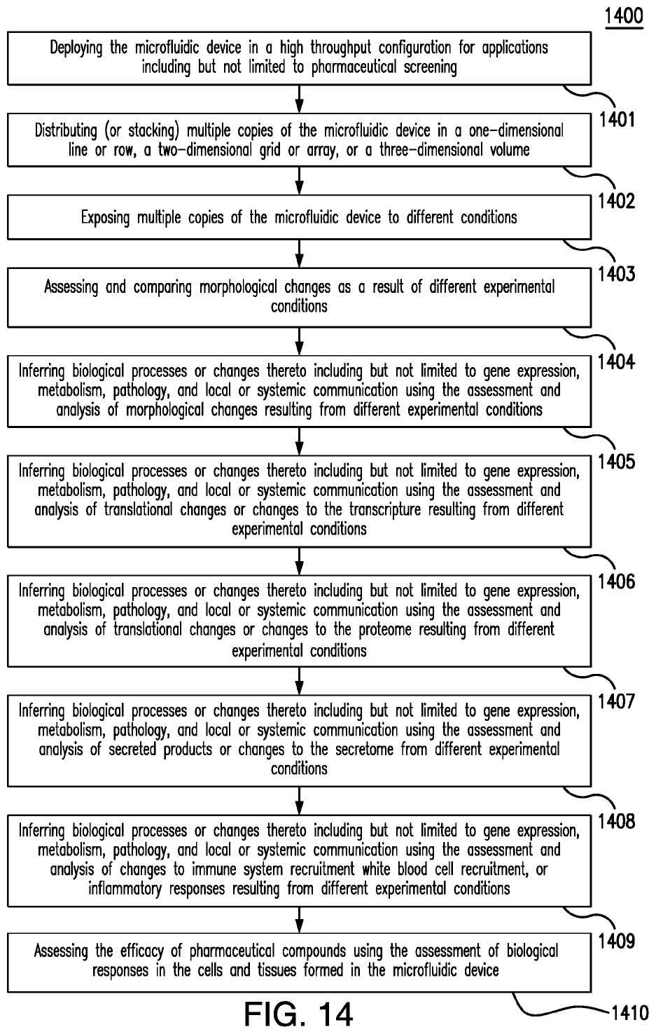

With reference to FIG. 14 for the purpose of illustration and not limitation, there is provided an exemplary method of deploying microfluidic device 1400 into high-throughput configurations, in accordance with the disclosed subject matter. For example, as shown in FIG. 14, a flow chart can describe subjecting one or more replicates to a first experimental condition, one or more additional replicates to a second experimental condition, and so forth. These conditions can include modifying any aspect of the subject matter that was discussed, including cell type, cell density, gel type, gel characteristics, device dimensions, layer thicknesses, and so forth, in accordance with the disclosed subject matter.

As shown in FIG. 14, the method shows at 1401 that the microfluidic device can be deployed in a high throughput configuration for applications including but not limited to pharmaceutical screening. At 1402, multiple copies of the microfluidic device can be distributed (or stacked) in a one-dimensional line or row, a two-dimensional grid or array, or a three-dimensional volume. At 1403, multiple copies of the microfluidic device can be exposed to different conditions. At 1404, morphological changes as a result of different experimental conditions can be assessed and compared. At 1405, the assessment and analysis of morphological changes resulting from different experimental conditions can be used to infer biological processes or changes thereto including but not limited to gene expression, metabolism, pathology, and local or systemic communication. At 1406, the assessment and analysis of transcriptional changes or changes to the transcriptome resulting from different experimental conditions can be used to infer biological processes or changes thereto including but not limited to gene expression, metabolism, pathology, and local or systemic communication. At 1407, the assessment and analysis of translational changes or changes to the proteome resulting from different experimental conditions can be used to infer biological processes or changes thereto including but not limited to gene expression, metabolism, pathology, and local or systemic communication. At 1408, the assessment and analysis of secreted products or changes to the secretome from different experimental conditions can be used to infer biological processes or changes thereto including but not limited to gene expression, metabolism, pathology, and local or systemic communication. At 1409, the assessment and analysis of changes to immune system recruitment, white blood cell recruitment, or inflammatory responses resulting from different experimental conditions can be used to infer biological processes or changes thereto including but not limited to gene expression, metabolism, pathology, and local or systemic communication. At 1410, the assessment of biological responses in the cells and tissues formed in the microfluidic device can be used to assess the efficacy of pharmaceutical compounds.

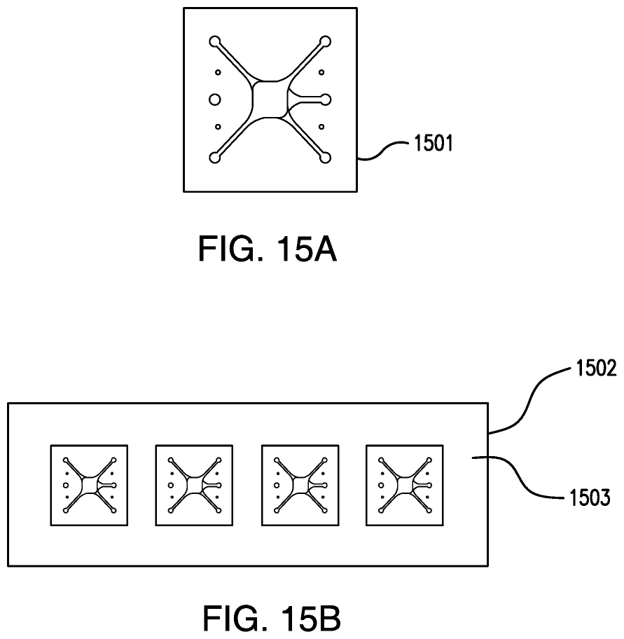

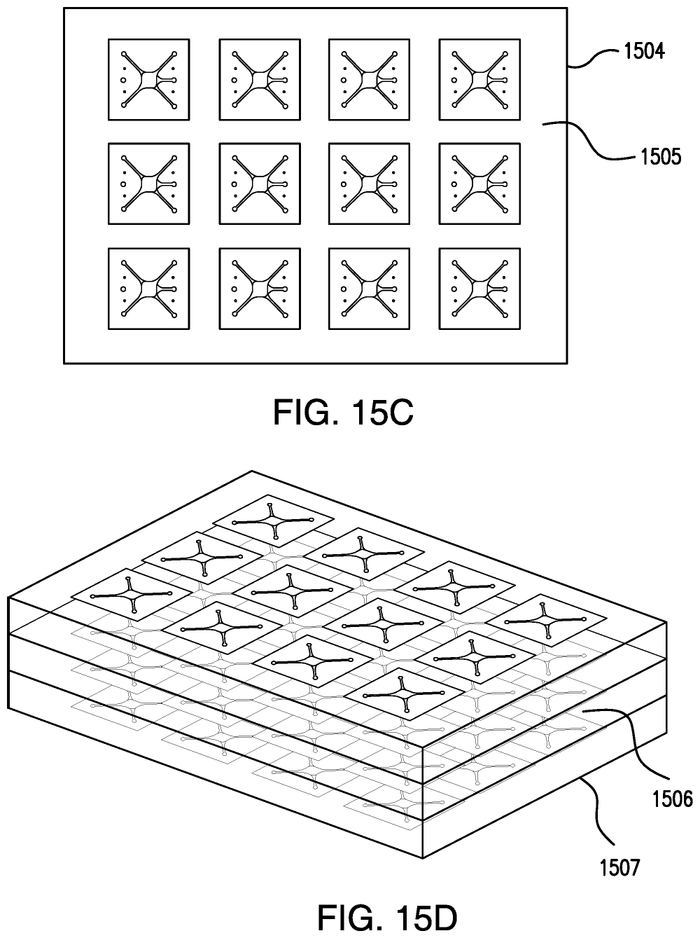

With reference to FIG. 15A for the purpose of illustration and not limitation, there is provided one replicate of an exemplary microfluidic device viewed top-down. At 1501 in FIG. 15A, a sample replicate of the microfluidic device viewed is illustrated.

With reference to FIG. 15B for the purpose of illustration and not limitation, there is provided multiple replicates of an exemplary microfluidic device deployed along a 1D row viewed top-down. At 1502 in FIG. 15B, an exemplary chamber, plate, or container to hold the one-dimensional row of replicates of the microfluidic device is illustrated. At 1503, an exemplary arrangement of replicates of the microfluidic device in a one-dimensional row arrangement is shown.

With reference to FIG. 15C for the purpose of illustration and not limitation, there is provided multiple replicates of an exemplary microfluidic device deployed along a 2D grid viewed top-down. At 1504 in FIG. 15C, an exemplary chamber, plate, or container to hold the two-dimensional grid of replicates of the microfluidic device is illustrated. At 1505, an exemplary arrangement of replicates of the microfluidic device in a two-dimensional row arrangement is shown.

With reference to FIG. 15D for the purpose of illustration and not limitation, there is provided multiple replicates of an exemplary microfluidic device stacked into a 3D array in a perspective view. At 1506 in FIG. 15D, an exemplary chamber, plate, or container to hold the three-dimensional stacked array of replicates of the microfluidic device is illustrated. At 1507, an exemplary arrangement of replicates of the microfluidic device in a three-dimensional stacked array arrangement is shown.

As a nonlimiting example intended for illustration, sample imaging by scanning electron microscopy requires a direct, unobstructed path to the biological sample, and thus cannot be performed on a biological tissue that is fully enclosed within a chamber that is opaque to electrons. Transmission electron microscopy requires that a sample be embedded in, and thinly cut from, a resin block approximately 3-5 mm in diameter, and thus also requires extraction of such a biological sample from within the chamber or chambers of a device that constitutes an exemplary specimen of the disclosed subject matter. For compatibility with the short working distances in high-magnification optical microscopy, the biological sample should typically be brought to within 1 mm or less from the objective lens, and thus in some embodiments of the disclosed subject matter that are not spatially compatible with this arrangement, the biological cells or tissues must also be physical extracted from their tissue culture chambers on the artificial airway device in order to be optically imaged in this configuration.

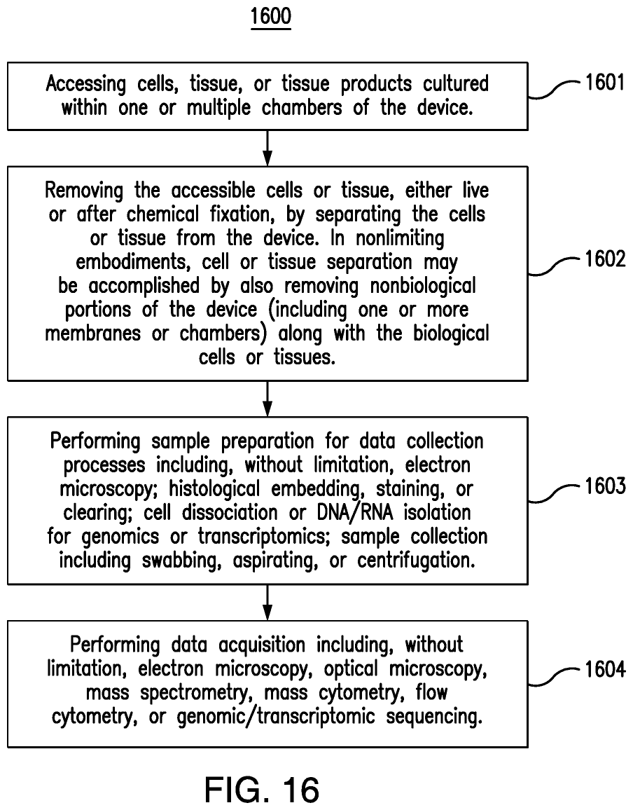

With reference to FIG. 16 for the purpose of illustration and not limitation, there is provided an exemplary method 1600 of extracting cells, tissue, or tissue products from a specimen of the disclosed subject matter, for purposes of performing data collection from, or obtaining measurements of, the cultured biological tissues. In a particular, nonlimiting embodiment, the cells, tissues, or tissue products are extracted from the disclosed subject matter following the method 1600 in order to achieve compatibility with imaging or data collection instrumentation. In an alternative and nonlimiting embodiment, in cases where a mechanism of measurement or data collection is already compatible with the disclosed subject matter, the physical extraction of cells, tissues, or tissue products from the airway device can still be performed in order to enhance, as one or a combination of the following examples without limitation, the measurement quality, measurement accuracy, sample quantity, sample mass, ease of collection, or biological sample integrity of the cells or tissue cultured therein.

As shown in FIG. 16, the method describes in 1601 that the partial or whole contents of the presently disclosed subject matter can be physically accessed in the device, and as shown in 1602, physically extracted from the device to the extent required for additional data collection, sample processing, or experimentation that is not compatible with the form factor of the microfluidic device. As additionally shown in 1602, in certain embodiments, the lung model device may be removed in whole or in part along with the extracted tissue. In some non-limiting embodiments, extraction of nonbiological device material in whole or in part along with the extracted cells, tissue, or tissue products can support the structural or spatial integrity of the extracted cells, tissue, or tissue products, can provide a substrate with which to physically grasp or manipulate the sample without altering the sample itself, or provide a combination thereof. As additionally shown in 1603, in certain embodiments, the sample extracted as described in 1602 may be processed according to the requirements of a data collection or measurement modality, including, without limitation, electron microscopy; histological embedding and sectioning, biological staining to create contrast or identify targets for analysis; isolation of genomic or transcriptomic cell products for genomic or transcriptomic analysis; direct sample collection utilizing tools including but not limited to swabs, biopsy punches, cell scrapers, or enzymatic dissociation products; or a combination thereof.

As a nonlimiting example of an exemplary process performed in certain embodiments of the subject matter, cells and tissues may have their RNA or DNA firstly stabilized with chemical preservatives, secondly physically extracted by pipetting, after which the remaining tissue is thirdly preserved with an electron microscopy chemical fixative, and fourthly postprocessed and imaged according to scanning electron microscopy protocols familiar to specialists in the field, in order to yield both transcriptomic data and electron microscopy topographic imagery. In some embodiments, such scanning electron microscopy protocols involve chemical fixation with glutaraldehyde, formaldehyde, cacodylate buffer, or a combination thereof, followed in certain embodiments by osmium tetroxide post-fixation, and followed in certain embodiments by carbon dioxide critical point drying and gold-palladium sputter coating. The method shown in 1604 describes without limitation the acquisition of data from the sample after the sample has been extracted and treated in a manner that is appropriate to the intended measurement and analysis workflow. As intended for explanation without limitation, in some embodiments in which a sample has been processed for electron microscopy imaging (by fixation, post-fixation, drying, and metal sputter coating), the sample is subsequently loaded onto an electron microscope stage and imaged. In other nonlimiting embodiments, a sample is treated or processed as required for data acquisition per 1603, and that acquisition modality or instrumentation is subsequently performed as described in 1604 in order to acquire the desired instrumentation data or biological measurements. Such modalities or instruments can include, without limitation, electron microscopy, optical microscopy, mass spectrometry, mass cytometry, flow cytometry, or genomic/transcriptomic sequencing.

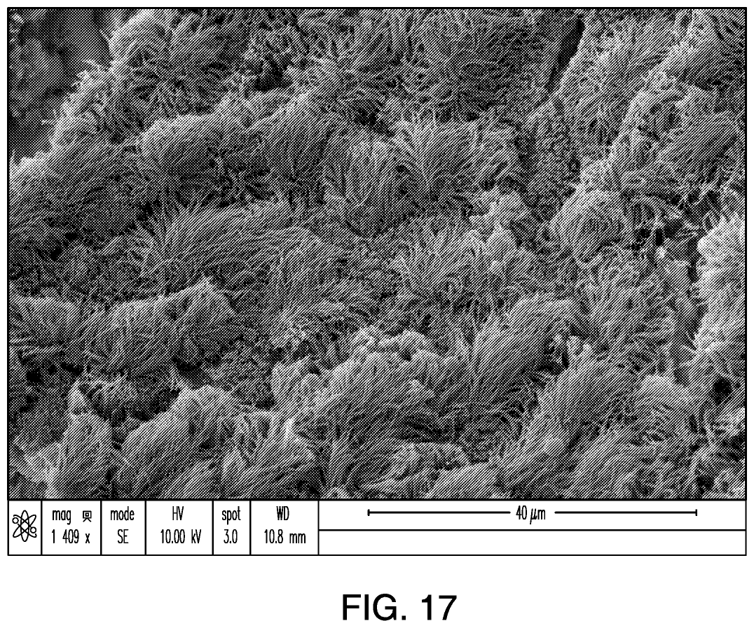

With reference to FIG. 17, for the purpose of illustration and not limitation, there is provided an exemplary scanning electron microscopy image of an exemplary differentiated epithelium of small airway cells, which display a high degree of filament-like ciliation on their apical, airway-facing surface. This widespread ciliation is indicative of a highly differentiated pulmonary epithelium as encountered in native human lungs. For purposes of illustration and not limitation, the tissue shown in FIG. 17 was extracted from an embodiment of the disclosed subject matter by peeling away the apical airway chamber in the lung model to access the epithelial cells dispensed for tissue culture upon a membrane within the device, four weeks prior to extraction. Following extraction, the isolated tissue shown in FIG. 17 was processed according to electron microscopy sample preparation protocols including glutaraldehyde-based fixation, osmium tetroxide-based post-fixation, carbon dioxide critical point drying, and gold-palladium sputtering to enhance surface conductivity. Following processing, the sample was loaded into a scanning electron microscope, and imaged to produce the exemplary data presented in FIG. 17.

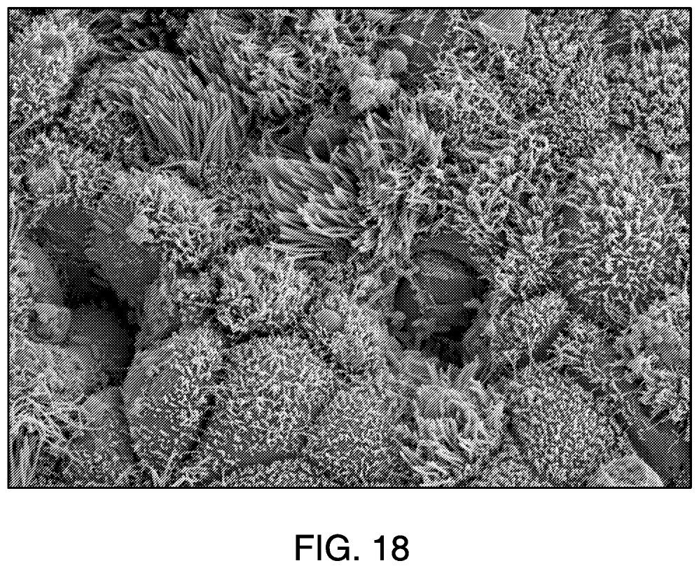

With reference to FIG. 18, for the purpose of illustration and not limitation, there is provided an exemplary scanning electron microscopy image of an exemplary differentiated epithelium of small airway cells, which were infected with bacteria two days prior to sample extraction. For purposes of illustration and not limitation, the tissue shown in FIG. 18 was extracted from an embodiment of the disclosed subject matter by peeling away the apical airway chamber in the lung model to access the epithelial cells and bacteria. Following extraction, the isolated tissue shown in FIG. 18 was processed according to electron microscopy sample preparation protocols including glutaraldehyde-based fixation, osmium tetroxide-based post-fixation, carbon dioxide critical point drying, and gold-palladium sputtering to enhance surface conductivity. Following processing, the sample was loaded into a scanning electron microscope, and imaged to produce the exemplary data presented in FIG. 18. This exemplary data illustrates without limitation that infection by Pseudomonas aeruginosa bacteria proceeds in the differentiated tissue cultured within the disclosed subject matter by a process that mimics infection by the same bacteria in living humans, beginning with digestion of epithelial tight junctions to form canyon- or crater-like passageways, through which the epithelium can be more easily degraded by attachment and attack by the bacteria on the more vulnerable basal surface.

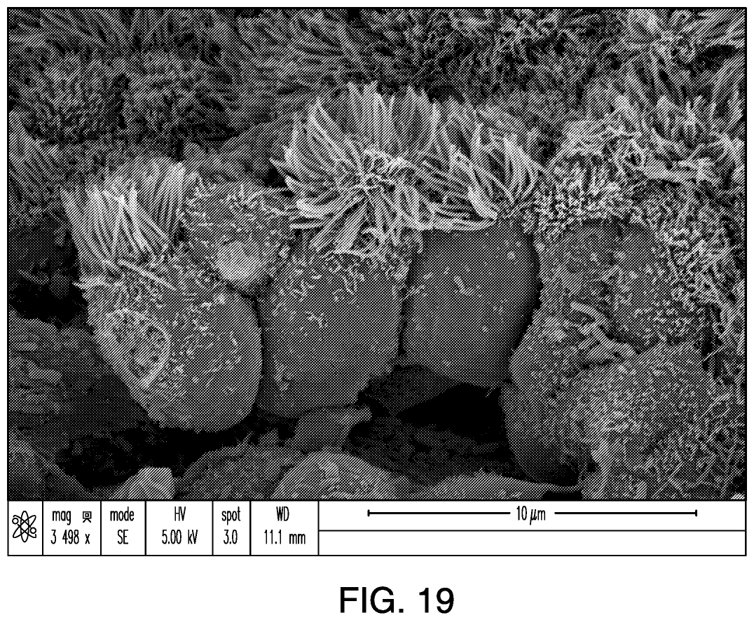

With reference to FIG. 19, for the purpose of illustration and not limitation, there is provided an exemplary scanning electron microscopy image of an exemplary differentiated epithelium of small airway cells, which were infected with bacteria two days prior to sample extraction. For purposes of illustration and not limitation, the tissue shown in FIG. 19 was extracted from an embodiment of the disclosed subject matter by peeling away the apical airway chamber in the lung model to access the epithelial cells and bacteria. Following extraction, the isolated tissue shown in FIG. 19 was processed according to electron microscopy sample preparation protocols including glutaraldehyde-based fixation, osmium tetroxide-based post-fixation, carbon dioxide critical point drying, and gold-palladium sputtering to enhance surface conductivity. Following processing, the sample was loaded into a scanning electron microscope, and imaged to produce the exemplary data presented in FIG. 19. This exemplary data illustrates without limitation the polarized differentiation observed in airway epithelial cells cultured in certain nonlimiting embodiments of the disclosed subject matter, which display a ciliated apical surface, and a flat basal surface from which a basement membrane is secreted.

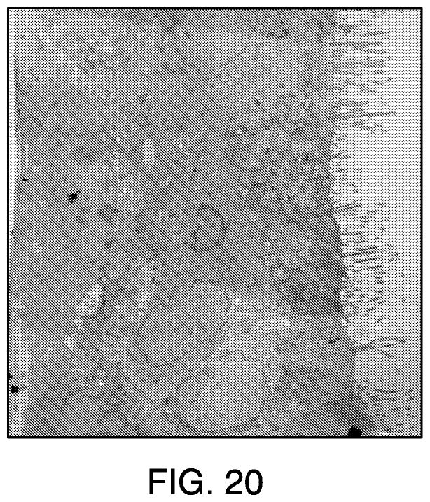

With reference to FIG. 20, for the purpose of illustration and not limitation, there is provided an exemplary transmission electron microscopy image of an exemplary differentiated epithelium of small airway cells. For purposes of illustration and not limitation, the tissue shown in FIG. 20 was extracted from an embodiment of the disclosed subject matter by peeling away the apical airway chamber in the lung model to access the epithelial cells and bacteria; this tissue was extracted and processed according to transmission electron microscopy sample preparation protocols, including fixation with solution composed of cacodylate buffer, glutaraldehyde, and formaldehyde, post-fixation with aqueous osmium tetroxide solution, embedding into epoxy resin, and sectioning on an ultramicrotome. Following processing, the sample was loaded into a transmission electron microscope, and imaged to produce the cross-sectional image shown in FIG. 20 for non-limiting illustration of the differentiated epithelial morphology observed in tissues cultured within a certain non-limiting embodiment of the disclosed subject matter.

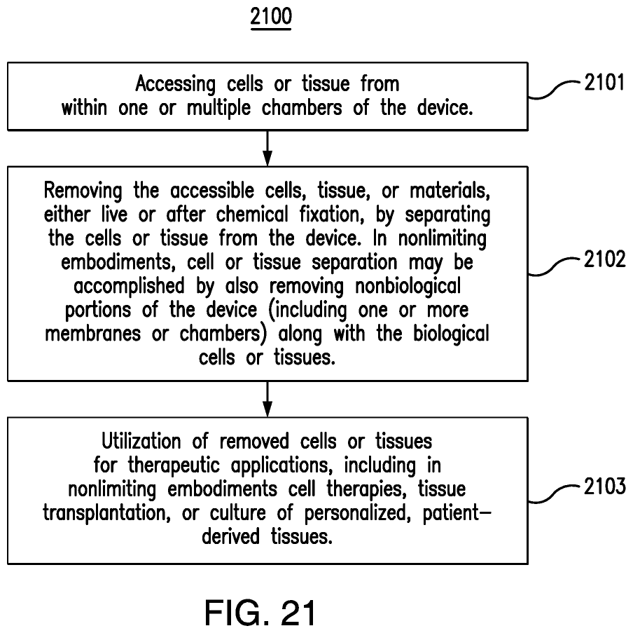

With reference to FIG. 21 for the purpose of illustration and not limitation, there is provided an exemplary method 2100 of extracting cells or tissue from a specimen of the disclosed subject matter, for therapeutic applications. In some embodiments, these therapeutic applications may include transplantation of the tissue cultured in the subject matter into living subjects. In some embodiments, transplantation of tissue extracted from the subject matter into living beings can be performed for wound healing and recovery. In some embodiments, the extracted cells can be processed to be delivered therapeutically by inhalation as an aerosol rather than by surgical transplantation. In some embodiments, the subject matter is used to culture or condition airway-associated or mesenchymal stem cells for stem cell therapies. In some embodiments, adult stem cells, embryonic stem cells, or patient-derived induced pluripotent stem cells can be cultured independently or in combination, and be extracted for therapeutic applications.

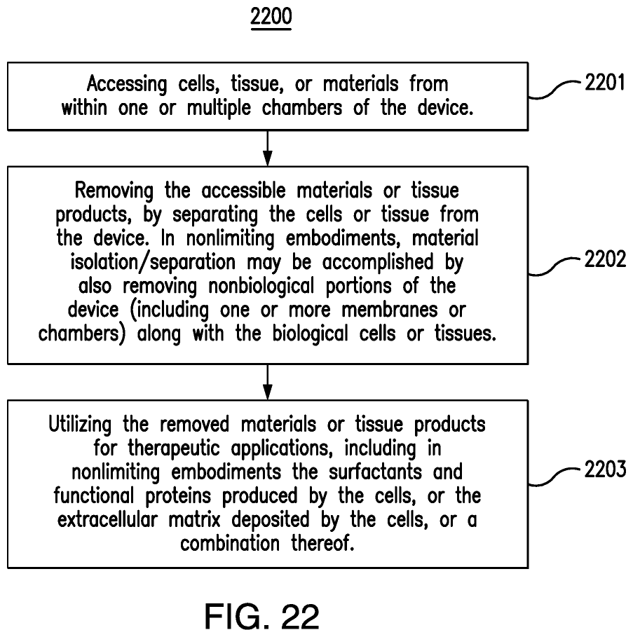

With reference to FIG. 22 for the purpose of illustration and not limitation, there is provided an exemplary method 2200 of extracting tissue products or materials from a specimen of the disclosed subject matter, for therapeutic applications. In certain nonlimiting embodiments, these therapeutic applications can include surfactants, proteins, hormones, extracellular matrix components, or secreted compounds that can be utilized as therapeutic compounds. In some embodiments, the subject matter serves as a bioreactor to manufacture these therapeutic compounds.

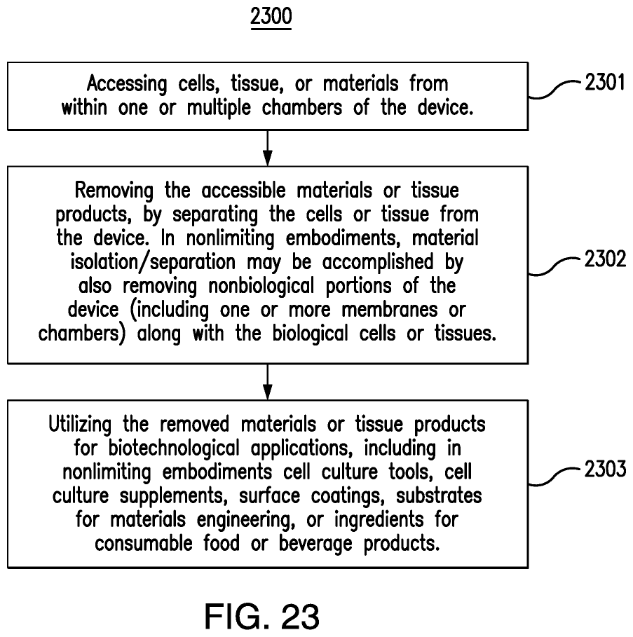

With reference to FIG. 23 for the purpose of illustration and not limitation, there is provided an exemplary method 2300 of extracting tissue products or materials from a specimen of the disclosed subject matter, for biotechnological or non-therapeutic applications. In certain nonlimiting embodiments, these extracted tissue products or materials can include surfactants, proteins, hormones, extracellular matrix components, secreted compounds, or a combination thereof. As nonlimiting examples, these extracted materials can be used for cell culture tools, cell culture supplements, cell culture substrates, surface coatings, reactants or substrates for materials engineering, or ingredients for consumable food or beverage products.

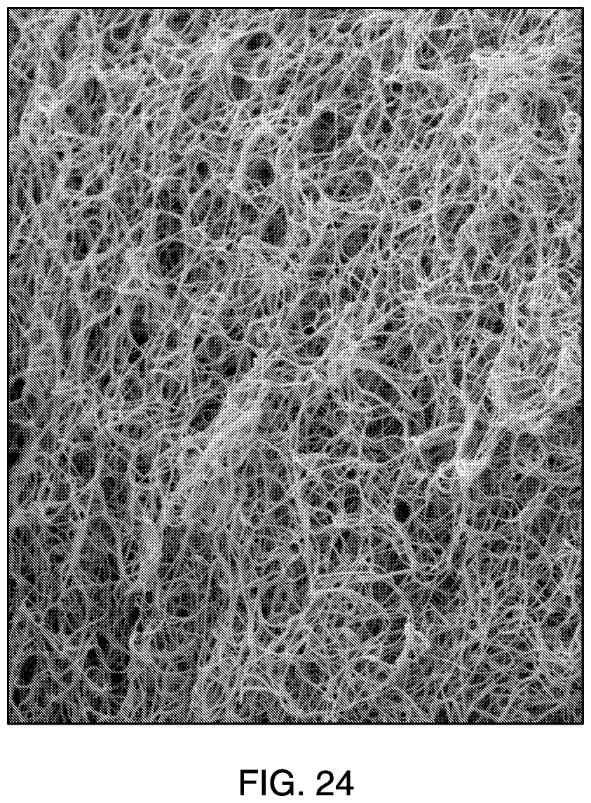

With reference to FIG. 24 for the purpose of illustration and not limitation, there is provided an exemplary transmission electron microscopy image of extracellular matrix extracted from the subject matter in accordance to the method provided in FIG. 23, after being cultured for four weeks following initial disposition of cells into the airway device. For purposes of illustration and not limitation, the extracellular matrix material depicted in FIG. 24 was extracted from an interstitial compartment in the airway device, by sequentially peeling the constitutive layers of a nonlimiting embodiment of the subject matter until the target extracellular matrix compartment was exposed, and then physically removing the extracellular matrix with laboratory forceps.

In certain embodiments, the chambers or channels of the subject matter can be accessed fluidically through open reservoirs. In certain embodiments, fluids can be added, removed, or sampled from the open reservoirs. In certain embodiments, fluid reservoirs can correspond to or be fluidically linked to one chamber. In certain embodiments, fluid reservoirs can correspond to or be fluidically linked to more than one chamber, to the limit given by the total number of chambers within the specific configuration of the subject matter to which the reservoir is linked.

In certain embodiments, chambers or channels of the subject matter can be accessed by additional microfluidic channels, tubing, or valving that acts to control the delivery of fluids—including but not limited to cell culture growth media, an aerosol, or air—to the chambers contained by the subject matter.

The presently disclosed subject matter provides, in part, a microfluidic device that can simulate the epithelial-stromal-endothelial multilayered architecture of the vascularized large and small human pulmonary airways, the transport or migration of compounds and cells across these layered interfaces, e.g., from the bloodstream, across the blood vessel endothelium, through the interstitium, and across the epithelium into the airway, and the dynamic behavior of the cells and materials from which these structures can be constituted. This microfluidic device can expand the capabilities of cell culture models, provide an alternative to certain animal or in vitro pulmonary models, and simulate the dynamics of disease states, bacterial or viral infection, and immune cell recruitment.

In certain embodiments, the microfluidic device includes a basal chamber, a first membrane, a central interstitial chamber, a three-dimensional extracellular matrix hydrogel, a second membrane, an apical chamber, a first monolayer of a first cell type, a second monolayer of a second cell type, and a third cell type that can be encapsulated within an extracellular matrix hydrogel. In certain embodiments, the basal chamber can have a first microfluidic channel disposed thereon. In certain embodiments, the interstitial chamber can have a second microfluidic channel disposed thereon. In certain embodiments, the apical chamber can have a third microfluidic channel disposed thereon. In certain embodiments, the interstitial chamber can be disposed between the basal chamber and apical chamber.

In certain embodiments, the first membrane can be disposed between the basal chamber and the interstitial chamber. In certain embodiments, the second membrane can be disposed between the interstitial chamber and apical chamber. In certain embodiments, the first membrane can have a first side and a second side. In certain embodiments, the first side of the first membrane can be oriented to face the basal chamber and the first monolayer of cells of a first cell type can be disposed on the first side of the first membrane. In certain embodiments, the second membrane can have a first side and a second side. In certain embodiments, the first side of the second membrane can be oriented to face the apical chamber and the second monolayer of cells of a second cell type can be disposed on the first side of the second membrane.

In certain embodiments in which the interstitial chamber is disposed between the basal and apical chambers, the second side of the first membrane and the second side of the second membrane can be oriented to face the interstitial chamber.

In certain embodiments, an extracellular matrix hydrogel can be disposed in the interstitial chamber between the first and second membranes.

In certain embodiments, cells of a third cell type can be encapsulated within the extracellular matrix hydrogel disposed between the first and second membranes.

In certain embodiments, the interstitial chamber is continuous between the first and second membranes such that fluid communication is permitted between the basal chamber and apical chamber via the interstitial chamber. In certain embodiments, bidirectional fluid communication and species transport is permitted from or to the basal chamber, through the first membrane, through interstitial fluid flow or diffusion through the extracellular matrix hydrogel in the interstitial chamber, and through the second membrane into the apical chamber. In certain embodiments, a fourth cell type can migrate through a membrane to pass from one chamber to another chamber. In certain embodiments with a first monolayer of a first type of cell disposed on the first side of a first membrane, a fourth cell type can migrate therethrough.

In certain embodiments, a fourth cell type can migrate through multiple membranes to move between multiple chambers. In certain embodiments including an extracellular matrix hydrogel disposed within an interstitial chamber that is disposed between a first membrane and subsequent basal chamber on the first side and a second membrane and subsequent apical chamber on the second side, a fourth cell type can migrate from the basal chamber through the first membrane and into the extracellular matrix hydrogel, and in certain embodiments the fourth cell type can continue to migrate from the extracellular matrix through the second membrane and into the apical chamber.

In certain embodiments in which at least one or more of a monolayer of a first cell type is disposed on a membrane, a first monolayer of a first cell type is disposed on a first membrane and a second monolayer of a second cell type is disposed on a second membrane, a fourth cell type can migrate therethrough to move from the first chamber to a second chamber, at least one or more of a fourth cell type can migrate from the first chamber through the first monolayer on the first membrane to a second chamber and subsequently from the second chamber through the second monolayer on the second membrane to a third chamber.

In certain embodiments, a membrane can be disposed between a first and second chamber, and the first chamber can contain air to create an air-liquid interface across the membrane. In certain embodiments, a membrane can be disposed between a first and second chamber, and the first chamber can have its liquid contents removed and substituted with air to create an air-liquid interface across the membrane. In certain embodiments, a monolayer of a second cell type can be disposed on the first side of a membrane that is oriented to face an apical chamber including air, to create a cellular interface between air in the apical chamber and liquid or extracellular matrix hydrogel in a second chamber on the second side of the membrane.

In certain embodiments, a third cell type can be encapsulated in the extracellular matrix hydrogel in an interstitial chamber that is adjacent to an air-liquid interface, e.g., to model stromal tissue adjacent to the air-facing pulmonary epithelium. In certain embodiments, an air interface can be formed in at least one of three or more chambers to create a as follows: an interstitial chamber can be disposed between a basal chamber and an apical chamber, with a first membrane separating the interstitial chamber from the basal chamber and a second membrane separating the interstitial chamber from the apical chamber, with a first monolayer of a first cell type disposed on the side of the first membrane facing the basal chamber, and a second monolayer of a second cell type disposed on the side of the second membrane facing the apical chamber, and with the interstitial chamber including a third cell type encapsulated in extracellular matrix hydrogel, such that when the apical chamber contains air, and the basal chamber contains fluid, e.g. cell growth medium or blood, the multi-chambered architecture models the epithelial, air-filled-stromal-endothelial, liquid-filled architecture of the large or small pulmonary airways.

In certain embodiments, the first cell type can be human umbilical vein endothelial cells (“HUVECs”). In certain embodiments, the first cell type can be human pulmonary microvascular endothelial cells (“HPMECs”). In certain embodiments, the first cell type can be human pulmonary endothelial cells isolated from human tissue. In certain embodiments, the first cell type can be arterial endothelial cells. In certain embodiments, the first cell type can be stem cell-derived endothelial cells. In certain embodiments, the second cell type can be human small airway epithelial cells (“HSAECs”). In certain embodiments, the second cell type can be human bronchial airway epithelial cells (“HBAECs”). In certain embodiments, the second cell type can be human tracheal airway epithelial cells (“HTAECs”). In certain embodiments, the second cell type can be stem-cell derived epithelial cells. In certain embodiments, the third cell type can be a human lung fibroblast cells (“HLFs”). In certain embodiments, the third cell type can be human mesenchymal stem cells (“hMSCs”). In certain embodiments, the third cell type can be human pericyte cells. In certain embodiments, the third cell type can be human cells isolated from stromal lung tissue. In certain embodiments, the third cell type can be human induced pluripotent stem cells (“iPSCs”). In certain embodiments, the fourth cell type can be human leukocytes (“white blood cells”). I n certain embodiments, the fourth cell type can be at least one of human neutrophils, eosinophils, basophils, lymphocytes, and macrophages, and a cell derived or differentiated therefrom. In certain embodiments, at least one of the first, second, and third cell type can be animal cells. In certain embodiments, at least one of the first, second, and third cell types can include an artificially or naturally induced pathology. In certain embodiments, at least one or more of the first, second, and third cell types can be isolated from diseased lungs. In certain embodiments, the naturally induced pathology can be from diseased animals or genetically engineered animal models of a disease.

In certain embodiments, the membranes can be porous polycarbonate membranes. In certain embodiments, the membranes can be porous polyester membranes. In certain embodiments, the membranes can be at least one or more of a polytetrafluoroethylene (PTFE) membrane, an elastomeric (e.g., polydimethylsiloxane) (PDMS), polyurethane) membrane, a paper membrane, and an extracellular matrix membrane (e.g., vitrified collagen). In certain embodiments, the pores of the membranes can be about 0.4 μm pores. In certain embodiments, the pores of the membranes can be about 50 μm pores. In certain embodiments, the pores can range from about 0.1 μm pores to about 1000 μm pores. In certain embodiments, the pores can have different sizes. In certain embodiments, the membranes can have different pore densities. In certain embodiments, each membrane's pore size can be selected to restrict the passage of entities (e.g., cells) of a larger size and allow passage only to entities physically smaller than the pore size (e.g., dissolved proteins). In certain embodiments, each membrane can possess multiple pore sizes with independently distributed densities, in order to differentially tune the transport characteristics of elements possessing different physical sizes.

In certain embodiments, a microchannel can be used to introduce a fluid to a basal, interstitial, or apical chamber. In certain embodiments, a microchannel can be used to perfuse or replace the fluid in a basal, interstitial, or apical chamber. In certain embodiments, a microchannel can be used to introduce one or more cell types to a basal, interstitial, or apical chamber. In certain embodiments, a microchannel can be used to introduce extracellular matrix hydrogel or one or more cell types encapsulated in an extracellular matrix hydrogel to one or more of a basal, interstitial, or apical chamber.

In accordance with certain embodiments of the disclosed subject matter, a method of fabricating a microfluidic device is provided. In certain embodiments, the method can include fabricating at least one or more of a basal chamber, an interstitial chamber, and an apical chamber. In certain embodiments, the basal chamber can have a first microfluidic channel disposed thereon. In certain embodiments, the interstitial chamber can have a second microfluidic channel disposed thereon. In certain embodiments, the apical chamber can have a third microfluidic channel disposed thereon. In certain embodiments, the method can include an interstitial chamber disposed between the basal chamber and apical chamber. In certain embodiments, the method can include a first membrane disposed between the basal chamber and the interstitial chamber. In certain embodiments, the method can include a second membrane disposed between the interstitial chamber and apical chamber.

In certain embodiments, the first membrane can have a first side and a second side. In certain embodiments, the method can include growing a first monolayer of cells of a first cell type on the first and/or second side of the first membrane, or cells of a first, second, third, or additional type on both sides of the first membrane. In certain embodiments, the method can include growing a first monolayer of cells of a first cell type on the first and/or second side of the first membrane, or cells of a first, second, third, or additional type on both sides of the first membrane. In certain embodiments, the second membrane can have a first side and a second side. In certain embodiments, the method can include growing a second monolayer of cells of a second cell type on the first and/or second side of the second membrane, or cells of a first, second, third, or additional type on both sides of the second membrane.

In certain embodiments in which the interstitial chamber is disposed between the basal and apical chambers, the second side of the first membrane and the second side of the second membrane can be oriented to face the interstitial chamber. In certain embodiments, an extracellular matrix hydrogel can be disposed in the interstitial chamber between the first and second membranes. In certain embodiments, cells of a third cell type can be encapsulated within the extracellular matrix hydrogel disposed between the first and second membranes. In certain embodiments, the method can include disposing an interstitial chamber that is continuous between the first and second membranes such that fluid communication is permitted between the basal chamber and apical chamber via the first membrane, the interstitial chamber, and the second membrane. In certain embodiments, the method can include fabricating a structure that permits bidirectional fluid communication and species transport from or to the basal chamber, through the first membrane, through interstitial fluid flow or diffusion through the extracellular matrix hydrogel in the interstitial chamber, and through the second membrane into the apical chamber.

In certain embodiments, growing the first monolayer of cells on the first membrane can include placing (e.g., flowing) the cells of the first cell type on the first side of the first membrane, creating a static environment to allow the cells to settle and attach to the membrane, and flowing a first culture medium over the cells of the first cell type. As used herein, the term “growing” involves the growth or replication of cells, or the culture or maintenance of cells such that they remain viable and representative of healthy or diseased human tissue, or representative of a phenotype or a morphology intended for the purpose of experimentation. In certain embodiments, growing the second monolayer of cells on the second membrane can include placing (e.g., flowing) the cells of the second cell type on the first side of the second membrane, creating a static environment to allow the cells to settle and attach to the membrane, and flowing at least one or more of a first and second culture medium over the cells of the second cell type. In certain embodiments, an extracellular matrix hydrogel is constituted by placing (e.g., flowing) a pre-gel solution into a first chamber, and permitting at least one of a gelation, curing, and hardening reaction to occur. In certain embodiments, an extracellular matrix hydrogel is formed by placing (e.g., at least one of flowing and injecting) a pre-gel solution into a first chamber, and exposing the pre-gel solution to a temporal stimulus including but not limited to at least one of elevated temperature and ultraviolet or visible light for photocatalysis, in order to induce at least one of gelation and curing of the pre-gel solution into a modified constitution (e.g., a crosslinked hydrogel). In certain embodiments, a third cell type can be encapsulated in extracellular matrix hydrogel by placing the cell type into a suspension with the pre-gel solution, placing (e.g. flowing or injecting) the pre-gel solution into a first chamber, and permitting a gelation, curing, or hardening reaction to occur, thereby producing the hydrogel encapsulation of the third cell type. In certain embodiments, a third cell type can be encapsulated in extracellular matrix hydrogel by placing the cell type into a suspension with the pre-gel solution, placing (e.g., flowing or injecting) the pre-gel solution into a first chamber, and exposing the pre-gel solution to a temporal stimulus including but not limited to elevated temperature or ultraviolet or visible light for photocatalysis, or a combination thereof, in order to induce gelation or curing of the pre-gel solution into a modified constitution (e.g., a crosslinked hydrogel).

In certain embodiments, one or more membranes can be used to spatially confine a pre-gel solution into a portion of the microfluidic device in order to produce a gel that is spatially patterned in a physiologically relevant manner. In certain embodiments, an extracellular matrix is spatially patterned in the chamber oriented to face the first side of a membrane that has a first and second side, such that elements on the second side of the membrane can be restricted from passing through the membrane pores even if the elements can be physically smaller, due to the pores being blocked by the extracellular matrix hydrogel in contact with the first side of the membrane. In certain embodiments, the surface tension of a pre-gel solution prevents the solution from leaking through the pores of an adjacent membrane, and thereby enables the spatial patterning of the hydrogel to only one side of the membrane. In certain embodiments, the surface tension of a pre-gel solution can prevent the pre-gel solution from passing through membrane pores of a larger diameter than a single cell (e.g., about 10 to about 100 μm pores), thereby allowing cells placed (e.g., flowed) over the pores on the first side of the membrane to be in direct contact with the extracellular matrix hydrogel that is placed on the second side of the membrane in regions where the cells pass through the pores of the membrane and settle upon the extracellular matrix hydrogel in contact with the second side of the membrane.

In certain embodiments, cell culture is maintained by placing the microfluidic device in a cell culture incubator. In certain embodiments, the microfluidic device is placed within a controllable atmosphere whose composition can be dynamically or statically adjusted at higher or lower levels of oxygen than normal at sea level. In other embodiments, the microfluidic device is placed at lower or higher levels of carbon dioxide than normal at sea level, ranging from 0% to 100%. In certain embodiments, the microfluidic device is placed within a controllable atmosphere whose pressure can be dynamically or statically adjusted e.g., to mimic the physiological conditions at high or low altitudes, or in overpressurized or depressurized environments. In certain embodiments, the microfluidic device can be operated at different flow rates to vary the hydrodynamic environment in the cell culture channels. In certain embodiments, the microfluidic device can be operated in zero-gravity, reduced-gravity, or increased-gravity conditions to mimic the behavior of tissues and cells contained therein in humans exposed to spaceflight or extraterrestrial environments with respect to nonstandard gravity. In certain embodiments, the microfluidic device can be operated in zero-gravity, reduced-gravity, or increased-gravity conditions to mimic the behavior of the tissues and cells contained therein in response to bacterial or viral infection of airway infections in humans exposed to spaceflight or extraterrestrial environments with respect to nonstandard gravity. In certain embodiments, the effluent can be disposed upon additional tissues or microfluidic models of tissues (e.g., cardiac tissues or cells, or liver tissues or cells) to simulate inter-tissue fluid communication. In certain embodiments, the pathologic secretions of the cells into the effluent of the microfluidic device as described hereinabove can be disposed upon additional tissues or microfluidic models of tissues (e.g., cardiac tissues or cells, or liver tissues or cells) to simulate inter-tissue fluid communication in a pathological state or disease state.

In accordance with certain embodiments of the disclosed subject matter, a method of testing airway tissue responses to airway infection is provided. In certain embodiments, this method can include providing a microfluidic device, as described hereinabove. In certain embodiments, this method can involve inoculating, with at least one of a fluid plug of bacteria or fluid suspension of bacteria, the apical chamber faced by a monolayer of epithelial cells that is disposed on a membrane. In certain embodiments, this method can involve inoculating, with an aerosolized stream of bacteria, the apical chamber faced by a monolayer of epithelial cells that is disposed on a membrane. In certain embodiments, inoculation with bacteria can be performed by placing (e.g., flowing) the bacteria through a microfluidic channel disposed on a chamber in the microfluidic device. In certain embodiments, inoculation with bacteria can be performed by injecting the bacteria into a chamber in the microfluidic device with at least one of a needle, cannula, and other such penetrative instrument which can gain access to one or more of the chambers in the microfluidic device. In certain embodiments, inoculation can be performed by partially or fully disassembling the microfluidic device to gain access to an otherwise sealed chamber. In certain embodiments, this method can include simulating physiological or pathological tissue conditions, by modifying aspects of the device including but not limited to membrane properties (including material composition, mechanical properties, pore sizes or pore densities, or thickness), tissue properties (including the use of diseased or healthy cell donors, concentrations of cells within healthy or diseased ranges), fluid composition (including concentrations in cell growth medium of inflammatory compounds or drug compounds which modify cell behavior or tissue properties), or air composition (e.g., clear air or air including smoke from a cigarette).

In non-limiting embodiments, the method can further include the placement of white blood cells into a first chamber, such that their response (e.g., migration) in response to the challenge of bacterial infection can be recorded. In certain embodiments, the method can include visualizing the behavior of the cells in the microfluidic device by non-limiting methods including microscopy. In certain embodiments, the method can include the discrete or continuous sampling of one or more secretory products from the cells (e.g., the concentration of one or more inflammatory cytokines, or the quantity of secreted mucus) or bacteria in the microfluidic device or one or more substances of interest in the microfluidic device.

In accordance with certain embodiments of the disclosed subject matter, a method of testing airway tissue responses to toxins or particulates is provided. In certain embodiments, this method can include providing a microfluidic device, as described hereinabove. In certain embodiments, this method can involve disposing (e.g., flowing) airborne or fluid-borne toxins or particulates into the apical, basal, or apical and basal chambers. In certain embodiments, the responses of one or a combination of the following tissues can be measured or monitored for acute injury or acute responses: the epithelial cells or tissues, the interstitial cells or tissues, or the endothelial cells or tissues. In certain embodiments, the responses of one or a combination of the following tissues can be measured or monitored for chronic injury or chronic pathogenesis: the epithelial cells or tissues, the interstitial cells or tissues, or the endothelial cells or tissues. In certain embodiments, measurement of cell health or cell responses is done by microscopy (e.g., phase imaging or fluorescent imaging). In certain embodiments, measurement of cell health or cell responses is done by sampling of the liquid or air effluent from the microfluidic device.

In accordance with certain embodiments of the disclosed subject matter, a method of analyzing the effects of DNA editing on airway tissues is provided. In certain embodiments, this method can include providing a microfluidic device, as described hereinabove. In certain embodiments, this method can involve disposing a mechanism of gene transfer or gene modification into the apical, interstitial, or basal chambers. In certain embodiments, the responses of cells to DNA editing in one or combination of the following tissues can be measured or monitored: epithelial cells or tissues, interstitial cells or tissues, or endothelial cells or tissues.

Although the presently disclosed subject matter and its advantages have been described in detail, it should be understood that various changes, substitutions and alterations can be made herein without departing from the spirit and scope of the disclosed subject matter as defined by the appended claims. Moreover, the scope of the present application is not intended to be limited to the particular embodiments described in the specification.

Patents, patent applications, publications, product descriptions, and protocols are cited throughout this application the disclosures of which are incorporated herein by reference in their entireties for all purposes.

1. A microfluidic device comprising:
a basal chamber, having a first microfluidic channel disposed thereon,
a first membrane, disposed on the basal chamber,
a central interstitial chamber, disposed on the first membrane and having a second microfluidic channel disposed thereon,
a second membrane, disposed on the central interstitial chamber,
an apical chamber, disposed on the second membrane and having a third microfluidic channel disposed thereon,
a three-dimensional, extracellular matrix hydrogel disposed in the central interstitial chamber, and
a base supporting the first, second and third microfluidic channels disposed therein.

2. The microfluidic device of claim 1, further comprising support pillars to prevent membrane deflection.

3. The microfluidic device of claim 1, wherein human primary lung fibroblast cells are encapsulated within the hydrogel.

4. The microfluidic device of claim 1, wherein the basal chamber is in fluid communication with the interstitial chamber through the first membrane.

5. The microfluidic device of claim 1, wherein the interstitial chamber is in fluid communication with the apical chamber through the second membrane.

6. The microfluidic device of claim 1, wherein the basal chamber, the interstitial chamber and the apical chamber are in fluid communication through the first membrane and the second membrane.

7. The microfluidic device of claim 1, wherein the interstitial chamber is continuous between the first and second membranes such that fluid communication is permitted between the basal chamber and the apical chamber via the interstitial chamber.

8. The microfluidic device of claim 1, wherein the first membrane has a first monolayer of endothelial cells disposed thereon, and the second membrane has a second monolayer of epithelial cells disposed thereon.

9. The microfluidic device of claim 1, wherein the first microchannel comprises a basal microfluidic inlet port and a basal chamber outlet port disposed thereon; the second microchannel comprises an interstitial chamber injection port disposed thereon; and the third microchannel comprises an apical microfluidic inlet port and an apical chamber outlet port disposed thereon, wherein the first, second, and third microchannels introduce a fluid to at least one or more of the basal, interstitial, and apical chambers.

10. The microfluidic device of claim 1, wherein bidirectional fluid communication and species transport is permitted from the basal chamber, through the first membrane, through the interstitial chamber, and through the second membrane into the apical chamber.

11. The microfluidic device of claim 1, further comprising multiple interstitial chambers stacked vertically to create layered structures reminiscent of stromal tissues in the lung.

12. The microfluidic device of claim 1, wherein the device further comprises one or more additional interstitial chambers, and a membrane between each two interstitial chambers.

13. The microfluidic device of claim 1, wherein the device further comprises one or more additional apical chambers bonded to the second membrane.

14. The microfluidic device of claim 1, wherein the device further comprises one or more additional basal chambers bonded to the first membrane.

15. The microfluidic device of claim 1, wherein the device comprises one or more additional interstitial chambers, and
wherein the intestinal chambers are layered directly on top of each other.

16. The microfluidic device of claim 15, wherein perfusable chambers are disposed between interstitial chambers containing hydrogels.

17. A microfluidic device comprising:
a basal chamber, having a first microfluidic channel disposed thereon,
a central interstitial chamber, on the basal chamber and having a second microfluidic channel disposed thereon,
an apical chamber, disposed on the central interstitial chamber and having a third microfluidic channel disposed thereon,
a three-dimensional, extracellular matrix hydrogel disposed in the central interstitial chamber, and
a base supporting the first, second, and third microfluidic channels disposed therein.

18. A method of fabricating a microfluidic device including a basal chamber, an interstitial chamber, and an apical chamber, the basal chamber having a first microfluidic channel disposed thereon, the interstitial chamber having a second microfluidic channel disposed thereon, and the apical chamber having a third microfluidic channel disposed thereon, comprising:
(a) disposing a first membrane between the basal chamber and the interstitial chamber;
(b) disposing a second membrane between the interstitial chamber and the apical chamber;
(c) placing cells encapsulated in a pre-gel solution into the interstitial chamber;
(d) allowing a first monolayer of cells to grow on the first membrane; and
(e) allowing a second monolayer of cells to grow on the second membrane.

19. The method of claim 18, further comprising adding one or more of a basal chamber, an interstitial chamber, an apical chamber, and a membrane at one or more interfaces between the basal chamber and the interstitial chamber, the interstitial chamber and a second interstitial chamber, and the interstitial chamber and the apical chamber.

20. A device fabricated by the method of claim 18.

21. A method of testing a bacterial infection of pulmonary airway and/or nasal cavity, the method comprising:
(a) providing the device of claim 20;
(b) placing bacteria in the apical chamber;
(c) allowing the bacteria to adhere to the second monolayer of cells, wherein the second layer of cells comprises epithelial cells.

22. A method of testing a viral infection of pulmonary airway and/or nasal cavity, the method comprising:
(a) providing the device of claim 20;
(b) placing viral capsids in the apical chamber;
(c) allowing the virus to infect one or more of the first monolayer of cells, the second monolayer of cells, or the cells in the interstitial chamber.

23. The method of claim 21, further comprising placing white blood cells into the basal chamber.

24. The method of claim 22, further comprising:
(a) monitoring white blood cell migration through the basal chamber, or the basal chamber and the interstitial chamber;
(b) monitoring interactions of white blood cells with the virus, or white blood cells with the bacteria.

25. The method of claim 23, further comprising inverting the device temporarily or permanently to permit or enhance white blood cell adhesion to the first membrane.

26. A method for modelling progression of a disease, a combination of diseases or a pathology of the airway and associated tissues, wherein the method comprises culturing patient-specific tissues or patient-specific cells in the device of claim 20,
wherein the patient-specific tissues or patient-specific cells are obtained from patients affected by the disease, the combination of diseases, or the pathology.

27. The method of claim 26, wherein the disease, or the pathology is selected from the group consisting of inflammation, age-related conditions, idiopathic conditions, genetic conditions, cell therapies, gene therapies, off-target drug effects, fibrosis, target drug effects, acute conditions, chronic conditions, and a combination thereof.

28. A method for modelling pathological effects on the airway and associated tissues caused by acute exposure, or chronic exposure, or acute and chronic exposure to radiation or contaminants, the method comprising:
(a) exposing the device of claim 20 to one or more of electromagnetic radiation, radiation of high-energy particles, radioactive materials, extraterrestrial materials, inorganic materials, organic materials, or a combination thereof;
(b) monitoring changes in the first monolayer of cells, the second monolayer of cells, and the cells in the interstitial chamber.

29. A method for developing functional artificial pulmonary systems, the method comprising:
(a) monitoring changes in the device of claim 20 caused by one or more of an environmental effect, a contaminant, a virus, bacteria, a disease, a pathology, or combinations thereof; and
(b) developing functional artificial pulmonary systems as full extracorporeal substitutes, partial extracorporeal substitutes, or extracorporeal models of pulmonary function for living subjects.
